(19) United States
(12) Patent Application Publication
(10) Pub. No.: US 2017/0252417 A1
(43) Pub. Date: Sep. 7, 2017

(54) PROTEIN-CHAPERONED T-CELL VACCINES

(71) Applicant: Massachusetts Institute of Technology, Cambridge, MA (US)

(72) Inventors: Darrell Irvine, Arlington, MA (US); Karl Dane Wittrup, Chestnut Hill, MA (US); Naveen K. Mehta, Cambridge, MA (US); Kavya Rakhra, Cambridge, MA (US); Eric Franklin Zhu, Lancaster, CA (US)

(21) Appl. No.: 15/452,266

(22) Filed: Mar. 7, 2017

Related U.S. Application Data
(60) Provisional application No. 62/304,697, filed on Mar. 7, 2016.

(51) Int. Cl. A61K 39/00; A61K 45/06; A61K 9/00; A61K 39/39; A61K 39/12; C12N 7/00

(52) U.S. Cl. CPC A61K 39/0011; A61K 39/12; C12N 7/00; A61K 9/0019; A61K 39/39; A61K 45/06; A61K 2039/53; C12N 2710/20071; C12N 2710/20034; A61K 2039/54; A61K 2039/577; A61K 2039/6081; A61K 2039/6031

(57) ABSTRACT

Protein antigens are provided. The protein antigens typically include a peptide antigen conjugated or fused to a chaperone protein to form a “chaperone-antigen” that increases lymph node uptake; improves an immune response; or a combination thereof relative to the peptide antigen alone. The immune response can be, for example, increased antigen-specific proliferation, enhanced cytokine production, stimulation of differentiation and/or effector functions, promotion of survival, rescue from exhaustion and/or anergy of T cells, or a combination thereof. Chaperon-antigens can also be used to induce tolerance and increase immune suppressive responses. In the most preferred embodiments, the peptide antigen is fused to the chaperone protein to form a fusion protein. The “chaperone-antigen” can be combined with an adjuvant to form a vaccine and administered to a subject to modulate an immune response to the antigen. Methods of increasing immune responses, treating cancer and infectious and inducing tolerance are also provided.

### CROSS-REFERENCE TO RELATED APPLICATIONS

This application claims benefit of U.S. Provisional Application No. 62/304,697, filed Mar. 7, 2016, which, where permissible, is specifically incorporated reference herein in its entirety.

### REFERENCE TO SEQUENCE LISTING

The Sequence Listing submitted Mar. 7, 2017 as a text file named “MIT18577H_ST25.txt,” created on Mar. 7, 2017, and having a size of 31,044 bytes is hereby incorporated by reference.

### STATEMENT REGARDING FEDERALLY SPONSORED RESEARCH

This invention was made with government support under Grant No. R01 CA174795 awarded by the National Institutes of Health. The government has certain rights in the invention.

### FIELD OF THE INVENTION

This invention relates to the field of vaccine technology, and more specifically to protein antigens which efficiently target the lymph nodes, and can be used to increase immune responses against the antigen.

### BACKGROUND OF THE INVENTION

The field of cancer immunotherapy is burgeoning with several clinical approvals in the past few years. However, cancer vaccines have lagged behind the clinical success of other strategies in immuno-oncology despite evidence indicating that cancer vaccines may synergize with other therapies such as checkpoint blockade inhibitors (Fu, et al., Cancer Res., 74(15):4042-4052 (2014)) and immunomodulatory cytokines (Schwartzentruber, et al., Cancer J., 17(5):343-350 (2011)). Sipuleucel-T, an autologous cellular vaccine against hormone therapy-resistant prostate cancer, remains to date the only FDA-approved cancer vaccine, although it provides only a modest survival benefit (Kantoff, et al., New Eng. J. Med., 363(5):412-22 (2010)). Furthermore, because it is based on manipulation of a patient's own immune cells, it suffers from logistical difficulties that provide barriers to its widespread adaptation. More logistically feasible peptide-based cancer vaccines, on the other hand, have historically provided response rates of merely 3-5% (Slingluff, Cancer J., 17(5): 343-350 (2011)). Thus, there is an urgent need to improve the potency of peptide vaccines.

It is an object of the invention to provide solutions for improving peptide vaccine trafficking from the site of injection to lymphoid organs, and compositions and methods of use thereof.

### SUMMARY OF THE INVENTION

Protein antigens and nucleic acids encoding them are provided. The protein antigens, typically including a peptide antigen conjugated or fused to a chaperone protein to form a “chaperone-antigen” that increases lymph node uptake; improves an immune response; or a combination thereof relative to the peptide antigen alone. The immune response can be, for example, increased antigen-specific proliferation of T cells, enhanced cytokine production by T cells, stimulated differentiation and/or effector functions of T cells, promotion of T cell survival, rescue from T cell exhaustion and/or anergy, or a combination thereof.

In some embodiments, the chaperone-antigen promotes a suppressive immune response or tolerance. In such embodiments, the peptide antigen is typically a self-antigen or another antigen to which tolerance is desired.

The protein chaperone can protect the antigen from degradation in vivo, thus increasing its half-life. The protein chaperone is typically either of sufficiently large molecular weight to facilitate effective lymph node uptake, or is a binders to endogenous molecules of sufficiently large molecular weight to do the same. The Examples below show that the protein chaperones can reduce or prevent a loss of potency of the antigen in the presence of the serum. Thus in some embodiments, the protein chaperone is a protein that when fused to an antigen of interest induces a stronger immune response in vivo than free antigen, even after incubation (e.g., overnight) with or otherwise in the presence of serum (e.g., 10% serum).

Typically, the size of the chaperone-antigen conjugate or fusion protein is at least about 45 kDa, 50 kDa, or larger. In some embodiments, the size of the chaperone component alone is at least about 45 kDa, 50 kDa, or larger. Preferably the protein chaperone is one that will not induce a systemic immune response in the subject. Thus the protein chaperone can be a protein that is endogenous to the subject to be treated, or a functional fragment or variant thereof. Exemplary protein chaperones include, but are not limited to, serum proteins such as albumins, globulins, fibrinogen, regulatory proteins, and clotting factors, and functional fragments and variants thereof.

In the most preferred embodiments, the peptide antigen is fused to the chaperone protein to form a fusion protein. The fusion protein can include a linking domain. The linking domain can include, for example, a first flexible linker linked to a purification tag linked to a second flexible linker. The linker can increase accumulation of the chaperone-antigen in the lymph node relative to a chaperone-antigen without the linker.

Peptide antigens are also provided. The peptide antigen can be, for example, derived from a virus, bacterium, parasite, plant, protozoan, fungus, tissue or transformed cell. The antigen can be neocancer antigen. The antigen can be a tolerizing or self-antigen.

Pharmaceutical compositions including the chaperone-antigen and a pharmaceutically acceptable carrier are provided. Vaccine compositions further including an adjuvant are also provided. In some embodiments, the adjuvant is selected from the group consisting of a lipidated CpG molecule, unformulated CpG, polyinosinic:polycytidylic acid (polyIC), and cyclic dinucleotides (CDN).

Methods of increasing an immune response and treating cancer and infectious diseases in a subject in need thereof are also provided. The methods typically include administering the subject an effective amount of chaperone-antigen or a nucleic acid encoding the chaperone antigen, optionally in combination with an adjuvant to increase an immune response in the subject.

Methods of increasing a suppressive immune response, or inducing or increasing tolerance in a subject in need thereof are also provided. The methods typically include administering the subject an effective amount of chaperone-antigen or a nucleic acid encoding the chaperone antigen, optionally in combination with an adjuvant to increase a suppressive immune response, or induce or increase tolerance in the subject.

The administration can be non-systemic. For example, the administration can be local. In particular embodiments, the administration is subcutaneous or intramuscular. The compositions can be administered as protein, or a nucleic acid encoding the protein which is expressed by subject's cells following administration.

An adjuvant can be in the same or a different pharmaceutical composition from the chaperone-antigen. In some embodiments, particularly tolerogenic embodiments, the compositions and methods can be carried out in the absence of an adjuvant.

Any of the methods can further include administering the subject an additional active agent. In some embodiments for treatment of cancer or infections, the additional active agent in an immunotherapeutic agent for example (i) a tumor targeting antibody, (ii) an extended serum half-life IL-2 (including, but not limited to, an serum albumin-IL2 such as MSA-IL2), (iii) an immune checkpoint inhibitor, or a combination thereof. The immune checkpoint can be mediated by, for example, PD-1 or CTLA-4. In some embodiments, the checkpoint inhibitor is a function blocking antibody.

### BRIEF DESCRIPTION OF THE DRAWINGS

FIG. 1 is a scheme of an exemplary mouse serum albumin (MSA) fusion construct including a peptide antigen fused to MSA. The C terminus of MSA is extended by a GGGS (SEQ ID NO: 13) linker, a His tag, another GGGS linker (SEQ ID NO: 13), and the peptide antigen of interest.

FIG. 2A is a schematic of a vaccine assay include priming at day zero (0), boost at day fourteen (14), and readout by tetramer stain analysis at day twenty (20). FIG. 2B is a dot plot showing % E7-tetramer+ of CD8+ T cells in mice vaccinated with 3 μg peptide equivalence of E7 long or MSA-E7 long fusion protein and adjuvant is cyclic dinucleotides (25 μg cyclic di-GMP) according to the vaccine assay of FIG. 2A. E7 is HPV E₃₇₋₅₈

(SEQ ID NO: 1)

(DGPAGQAEPDRAHYNIVTF).

FIGS. 2C-2E are bar graphs showing % E7-tetramer+ of CD8+ T cells in mice vaccinated with 3 μg peptide equivalence of E7 long or MSA-E7 long fusion protein and adjuvant: 50 μg poly(C) (FIG. 2C), 1.23 nmol CpG (FIG. 2D), or 1.23 nmol lipo-CpG (FIG. 2E).

FIG. 3A is a schematic of a vaccine assay include priming at day zero (0), boost at day thirteen (13), and readout by intracellular cytokine staining analysis at day nineteen (19). FIGS. 3B-3C are dot plots showing % E7-tetramer+ of CD8+ T cells in mice vaccinated with 3 μg peptide equivalence of E7 long, Fc-E7 long fusion, or sso7d-E7 long fusion along with 25 μg cyclic di-GMP according to the vaccine assay of FIG. 3A. FcWT is the Fc portion of mouse IgG2a and FcKO is a mutant form of the Fc portion of mouse IgG2a (G236R and L328R) that abrogates interaction with Fcγ receptors (FIG. 3B); and M11.1.2 and M18.2.5 are two variants MSA-binding variants of an archae-derived DNA-binding protein named sso7d. FIG. 3D is a bar graph showing % response of CD8+ T cells in mice vaccinated with 25 μg cyclic di-GMP adjuvant and free Trp1, MSA-Trp1 antigen fusion, free EGP, MSA-EGP antigen fusion, free CEA, or MSA-CEA antigen fusion (FIG. 3D). In the case of Trp1, an altered peptide ligand form of the Trp1 antigen (where the C-terminal anchor residue A is replaced with M) was utilized. Trp1—vaccine: TAPDNLGYM (SEQ ID NO:3); Restimulation: TAPDNLGYA (SEQ ID NO:4). CEA—vaccine:

(SEQ ID NO: 2)

RAYVSGIQNSVSANRSDP;

### Restimulation:

(SEQ ID NO: 2)

RAYVSGIQNSVSANRSDP.

EGP is also the name of an altered peptide sequence of a melanocyte-associated antigen named gp100 native sequence: AVGALEGSRNQDWLGVPRQL (SEQ ID NO:20), altered sequence: AVGALEGPRNQDWLGVPRQL (SEQ ID NO:21).

FIG. 4A is a dot plot showing Radiant Efficiency [p/s/cm2/sr]/[μW/cm] of labeled MSA-E7 long (6 μg dose) and E7 long peptide alone (6 μg, 15 μg, 30 μg, 60 μg dose) in mouse lymph nodes. FIG. 4B is a dot plot showing Radiant Efficiency [p/s/cm2/sr]/[μW/cm] of labeled MSA-E7 long (6 μg dose). FcWT-E7 long, FcKO-E7 long, and soluble E7 long (3 μg peptide equivalent) in mouse lymph nodes.

FIG. 5A is a diagram illustrating strategies for multifactorial stimulation of the anti-tumor immune response. Adapted from Marcia Belvin and Ira Mellman, Sci Transl Med, 7:315-48 (2015). FIG. 5B is a tumor growth curve (tumor area (mm²)) showing the results of combination immunotherapy (untreated, PD1, CTLA4, or PD1+CTLA4) in a 2677-CEA tumor model (n=5). FIG. 5C is plot showing individual tumor growth curves (tumor area (mm²)) of mice that have regressed tumors. FIG. 5D is a plot showing cumulative survival of tumor bearing mice. Death events correspond to tumors of area >150 mm². FIG. 5E is a plot showing mouse body weight tracked over time (n=5). For FIGS. 5B-5E arrows along the x-axis indicate points of administration of 5 weekly treatments of the combination immunotherapy, beginning on day 6. FIG. 5F is a Kaplan-Meier curve showing the effect of PBS (vehicle), cyclic dinucleotide adjuvant alone (CND), cyclic dinucleotide adjuvant in combination with free E7 long peptide antigen (E7 long Peptide+CDN), and cyclic dinucleotide adjuvant in combination with MSA-E7 long peptide antigen fusion protein (MSA-E7 long Peptide+CDN).

FIG. 6A is a bar graph showing % E7-tetramer+ of CD8+ T cells in mice vaccinated with TTR-E7 long fusion protein or MSA-E7 long fusion protein. Because TTR is a tetramer, there are four copies of E7 long peptide cargo per protein. As a result, 25 μg of TTR-E7 long were compared against 100 μg MSA-E7 long in a prime boost vaccination model. FIGS. 6B-6D are bar graphs showing % response of CD8+ T cells following vaccination with TTR-Trp1 or MSA-Trp1 (FIG. 6B), TTR-EGP long or MSA-EGP (FIG. 6C), and TTR-CEA long or MSA-CEA long (FIG. 6D). FIGS. 6E and 6F are bar graphs showing the % response of CD8+ T cells following vaccination with TTR-EGP long fusion protein, TTR-Trp1 fusion protein, TTR-EGP long fusion protein and TTR-Trp1 fusion protein, TTR-EGP long-Trp1 fusion protein, or TTR-Trp1-EGP long fusion protein with Trp1 restimulation (FIG. 6E) or EGP restimulation (FIG. 6F).

FIG. 7 is a bar graph comparing % E7-tetramer+ of CD8+ T cells in mice vaccinated with MSA-E7 long fusion protein or dendritic cell-targeted DEC1-MSA-E7-long fusion protein.

FIGS. 8A and 8B are curves showing the % of injected dose of free E7 long peptide (FIG. 8A) and MSA-E7 long fusion protein (FIG. 8B) in blood following either intravenously (IV) or subcutaneously (SQ) into mice. FIGS. 8C and 8D are curves showing normalized response of CD8+ T cells restimulated with a dilution series of free E7 long peptide (FIG. 8A) or MSA-E7 long fusion protein (FIG. 8B), either fresh from the fridge (“fresh”) or following overnight treatment with 10% mouse serum at 37 C (“serum treated”).

FIG. 9A is a schematic of a tolerizing vaccine assay include priming (tolerizing) at day zero (0), boost (tolerizing) at day fourteen (14), prime (challenge) at day forty-five (45), boost (challenge) at day fifty-nine (59), and tetramer stain at day sixty-five (65). FIG. 9B is a bar showing % E7-tetramer+ of CD8+ T cells in mice treated with free E7 long, MSA-E7 long, or PBS, according to the tolerizing vaccine assay of FIG. 9A.

FIG. 10 is a bar showing % E7-tetramer+ of CD8+ T cells in mice treated with pVax-E7 long (plasmid) DNA vaccine, or pVax-MSA-E7 long (plasmid) DNA vaccine.

FIG. 11 is a bar graph showing nmol/L if culture of MSA-E7 long chaperone-antigen protein and free E7 protein expression in transfect HEK cells.

### DETAILED DESCRIPTION OF THE INVENTION

### I. Definitions

As used herein, “immunostimulatory oligonucleotide” is an oligonucleotide that can stimulate (e.g., induce or enhance) an immune response.

As used herein, “CG oligodeoxynucleotides (CG ODNs)” are short single-stranded synthetic DNA molecules that contain a cytosine nucleotide (C) followed by a guanine nucleotide (G).

As used herein, “immune cell” is meant a cell of hematopoietic origin and that plays a role in the immune response. Immune cells include lymphocytes (e.g., B cells and T cells), natural killer cells, and myeloid cells (e.g., monocytes, macrophages, eosinophils, mast cells, basophils, and granulocytes).

As used herein, the terms “immune activating response”, “activating immune response”, and “immune stimulating response” refer to a response that initiates, induces, enhances, or increases the activation or efficiency of innate or adaptive immunity. Such immune responses include, for example, the development of a beneficial humoral (antibody mediated) and/or a cellular (mediated by antigen-specific T cells or their secretion products) response directed against a peptide in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response can also involve activation of monocytes, macrophages, NK cells, basophils, dendritic cells, astrocytes, microglia cells, eosinophils, activation or recruitment of neutrophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

As used herein, the terms “suppressive immune response” and “immune suppressive response” refer to a response that reduces or prevents the activation or efficiency of innate or adaptive immunity.

As used herein, the term “immune tolerance” as used herein refers to any mechanism by which a potentially injurious immune response is prevented, suppressed, or shifted to a non-injurious immune response (Bach, et al., N. Eng. J. Med., 347:911-920 (2002)).

As used herein, the term “tolerizing vaccine” as used herein is typically an antigen-specific therapy used to attenuate autoreactive T and/or B cell responses, while leaving global immune function intact.

As used herein, the term “immunogenic agent” or “immunogen” is capable of inducing an immunological response against itself on administration to a mammal, optionally in conjunction with an adjuvant.

As used herein, the term “immune cell” refers to cells of the innate and acquired immune system including neutrophils, eosinophils, basophils, monocytes, macrophages, dendritic cells, lymphocytes including B cells, T cells, and natural killer cells.

As used herein, the term “T cell” refers to a CD4+ T cell or a CD8+ T cell. The term T cell includes TH1 cells, TH2 cells and TH17 cells.

As used herein, the term “T cell cytoxicity” includes any immune response that is mediated by CD8+ T cell activation. Exemplary immune responses include cytokine production, CD8+ T cell proliferation, granzyme or perforin production, and clearance of an infectious agent.

As used herein, the terms “peptide,” “polypeptide,” and “protein” refer to a chain of amino acids of any length, regardless of modification (e.g., phosphorylation or glycosylation). The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the terms “effective amount” or “therapeutically effective amount” means a dosage sufficient to provide treatment for a disorder, disease, or condition being treated, to induce or enhance an immune response, or to otherwise provide a desired pharmacologic and/or physiologic effect. The precise dosage will vary according to a variety of factors such as subject-dependent variables (e.g., age, immune system health, etc.), the disease, the disease stage, and the treatment being effected.

As used herein, “oligonucleotide” or a “polynucleotide” are synthetic or isolated nucleic acid polymers including a plurality of nucleotide subunits.

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

As used herein, the term “identity,” as known in the art, is a relationship between two or more polypeptide sequences, as determined by comparing the sequences. In the art, “identity” also means the degree of sequence relatedness between polypeptide as determined by the match between strings of such sequences. “Identity” can also mean the degree of sequence relatedness of a polypeptide compared to the full-length of a reference polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M, and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein, the phrase that a molecule “specifically binds” or “displays specific binding” to a target refers to a binding reaction which is determinative of the presence of the molecule in the presence of a heterogeneous population of other biologics.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences.

As used herein, an “expression control sequence” is a DNA sequence that controls and regulates the transcription and/or translation of another DNA sequence.

As used herein, the term “host cell” refers to prokaryotic and eukaryotic cells into which a recombinant nucleotide, such as a vector, can be introduced.

As used herein, “transformed” and “transfected” encompass the introduction of a nucleic acid (e.g. a vector) into a cell by a number of techniques known in the art.

As used herein, the phrase “operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, the term “carrier” refers to an organic or inorganic ingredient, natural or synthetic, with which the active ingredient is combined to facilitate the application.

As used herein, the term “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, the term “pharmaceutically-acceptable carrier” means one or more compatible solid or liquid fillers, dilutants or encapsulating substances which are suitable for administration to a human or other vertebrate animal.

As used herein, the term “treating” includes inhibiting, alleviating, preventing or eliminating one or more symptoms or side effects associated with a disease or disorder.

As used herein, the term “reduce”, “inhibit”, “alleviate” or “decrease” are used relative to a control. One of skill in the art would readily identify the appropriate control to use for each experiment.

As used herein, the terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

As used herein, the term “endogenous” means substances and processes that originate from within an organism, tissue, or cell.

### II. Compositions

One problem that peptide vaccines commonly face is their inability to efficiently traffic from the site of injection to secondary lymphoid organs. Thus, strategies for improving the potency of peptide antigens using protein chaperones, and to increase bioavailability in the lymph node following administration, particularly by subcutaneous injection, are provided.

Although proteins carriers have been proposed to deliver immunogens, typically protein-antigen conjugates are implemented for the purposes of physically anchoring antigens to CD4-epitope containing carriers—such as keyhold limpet hemocyanin, bovine serum albumin, or ovalbumin—to induce humoral immunity (Musselli, et al., J. Cancer Res Clin. Oncol., 127(suppl 2):R20-26 (2001). Alternatively, investigators have attached small molecular components including haptens and peptides to larger proteins to improve circulating half-life. As discussed in more detail below, the disclosed fusion peptides typically include a T cell epitope or T cell antigen fused to a protein chaperone. The compositions can be administered subcutaneously, avoid circulatory uptake, target the lymph node, and generate cellular immunity to the peptide antigens.

Pharmaceutical compositions can include a protein chaperone-antigen fusion protein or conjugate alone or in combination with an adjuvant. In some embodiments, an adjuvant is present in a second composition.

The fusion protein, the adjuvant, or a combination thereof can be packaged into a nanoparticulate delivery system.

A. Immunizing Proteins

Immunizing proteins for use in peptide vaccines are provided. The proteins typically include a protein chaperone (also referred to herein a protein carrier) and an antigen.

The antigen and the chaperone are covalently or non-covalently linked to form a “chaperone-antigen.” The chaperone-antigens optionally include an intervening linker sequence. In preferred embodiments, the chaperone-antigen is a fusion protein.

The fusion proteins can have a first fusion partner including a peptide antigen fused (i) directly to a protein chaperone or, (ii) optionally, fused to a linker peptide sequence that is fused to the protein chaperone. The fusion proteins optionally contain a domain that functions to dimerize or multimerize two or more fusion proteins. The peptide/polypeptide linker domain can either be a separate domain, or alternatively can be contained within one of one of the other domains (peptide antigen or protein chaperone) of the fusion protein. Similarly, the domain that functions to dimerize or multimerize the fusion proteins can either be a separate domain, or alternatively can be contained within one of one of the other domains (peptide antigen, protein chaperone or peptide/polypeptide linker domain) of the fusion protein. In one embodiment, the dimerization/multimerization domain and the peptide/polypeptide linker domain are the same.

Fusion proteins disclosed herein can be of formula I:

N—R₁—R₂—R₃—C

wherein “N” represents the N-terminus of the fusion protein, “C” represents the C-terminus of the fusion protein, “R₁” is a protein chaperone, “R₂” is an optional peptide/polypeptide linker domain, and “R₃” is a peptide antigen. Alternatively, R₃ may be the protein chaperone and R₁ may be the peptide antigen.

The fusion proteins can be dimerized or multimerized. Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric.

In some embodiments, the fusion protein includes two or more antigens. The antigens can be directly adjacent, or separated by the chaperone, a linker, or another domain. Thus in some embodiments, the chaperone has the same or different antigens fused to both the N-terminal and C-terminal ends, optionally with linkers. In some embodiments, two or more antigens are fused in tandem, optionally with linkers between them, to the N-terminal end, the C-terminal end, or both, of the chaperone.

1. Antigens

The immunizing proteins include an antigen. A suitable antigen can be selected based on the desired therapeutic outcome and the disease, disorder, or condition being treated. The disclosed compositions are exemplified in the Examples below as fusion proteins that include, as a peptide antigen, a peptide fragment derived from HPV (HPV E7₃₈₋₅₇ (IDGPAGQAEPDRAHYNIVTF (SEQ ID NO: 1)), human carcinoembryonic antigen (CEA)₅₆₇₋₅₈₄

(SEQ ID NO: 2)

(RAYVSGIQNSVSANRSDP),

or altered peptide ligand forms of mouse tyrosinase-related protein 1 (Trp1)₄₅₅₋₄₆₃ (TAPDNLGYM (SEQ ID NO:3) or TAPDNLGYA (SEQ ID NO:4)).

Functional fragments and variants of SEQ ID NOS: 1-4 are also provided. A particular fragment of SEQ ID NO: 1 is DGPAGQAEPDRAHYNIVTF (SEQ ID NO:5). In other embodiments, the peptide antigen is a variant or functional fragment of any of SEQ ID NOS: 1-5 having at least 50, 60, 70, 75, 80, 85, 90, 95, 96, 97, 98, 99 percent sequence identity to SEQ ID NO: 1-5. A functional fragment or variant can be one that induces an immune response against, particularly when administered along with a chaperone protein as a chaperone-antigen conjugate or fusion protein.

However, the foregoing antigens are exemplary in nature, and it will be appreciated that the principles can also be applied more generally. The antigen can be derived from a virus, bacterium, parasite, plant, protozoan, fungus, tissue or transformed cell such as a cancer or leukemic cell.

Suitable antigens are known in the art and are available from commercial government and scientific sources. The antigens may be purified or partially purified polypeptides derived from tumors or viral or bacterial sources. The antigens can be recombinant polypeptides produced by expressing DNA encoding the polypeptide antigen in a heterologous expression system.

Antigens may be provided as single antigens or may be provided in combination. In some embodiments, the conjugate or fusion protein includes 2, 3, 4, or more peptide antigens. In some embodiments, an antigen is between about 5 and about 50 amino acids, or between about 7 and 40 amino acids, or between about 12 and 25 amino acids, or between about 9 and 20 amino acids in length. In some embodiments, the antigen is more than 50 amino acids. In some embodiments, the antigen is at least 50 kDa. In some embodiments, the peptide antigen is a between about 500 Da and about 50,000 Da, or between about 500 Da and about 25,000 Da, or between about 500 Da and about 10,000 Da, or between about 500 Da and about 5,000 Da, or about 500 Da and about 1,000 Da, or between about 1,000 Da and about 10,000 Da, or between about 1,000 Da and about 25,000 Da, or between about 1,000 Da and about 50,000 Da. Exemplary sources of antigens are provided below.

a. Viral Antigens

A viral antigen can be isolated from and or derived from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens may be derived from a particular strain such as a papilloma virus, a herpes virus, e.g., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

b. Bacterial Antigens

Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

c. Parasite Antigens

Parasite antigens can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

d. Cancer Antigens

The antigen can be a cancer antigen. A cancer antigen is an antigen that is typically expressed preferentially by cancer cells (i.e., it is expressed at higher levels in cancer cells than on non-cancer cells) and in some instances it is expressed solely by cancer cells. The cancer antigen may be expressed within a cancer cell or on the surface of the cancer cell. The cancer antigen can be MART-1/Melan-A, gp100, adenosine deaminase-binding protein (ADAbp), FAP, cyclophilin b, colorectal associated antigen (CRC)—C017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-1, GAGE-2, GAGE-3, GAGE-4, GAGE-5, GAGE-6, GAGE-7, GAGE-8, GAGE-9, BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100Pmel117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, or c-erbB-2.

e. Allergens and Environmental Antigens

The antigen can be an allergen or environmental antigen, such as, but not limited to, an antigen derived from naturally occurring allergens such as pollen allergens (tree-, herb, weed-, and grass pollen allergens), insect allergens (inhalant, saliva and venom allergens), animal hair and dandruff allergens, and food allergens. Important pollen allergens from trees, grasses and herbs originate from the taxonomic orders of Fagales, Oleales, Pinales and platanaceae including i.e. birch (Betula), alder (Alnus), hazel (Corylus), hornbeam (Carpinus) and olive (Olea), cedar (Cryptomeria and Juniperus), Plane tree (Platanus), the order of Poales including i.e. grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other allergen antigens that may be used include allergens from house dust mites of the genus Dermatophagoides and Euroglyphus, storage mite e.g. Lepidoglyphys, Glycyphagus and Tyrophagus, those from cockroaches, midges and fleas e.g. Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

f. Tolerogenic Antigens

The antigen can be a tolerogenic antigen. Exemplary antigens are known in the art. See, for example, U.S. Published Application No. 2014/0356384.

In some cases, the tolerogenic antigen is derived from a therapeutic agent protein to which tolerance is desired. Examples are protein drugs in their wild type, e.g., human factor VIII or factor IX, to which patients did not establish central tolerance because they were deficient in those proteins; or nonhuman protein drugs, used in a human. Other examples are protein drugs that are glycosylated in nonhuman forms due to production, or engineered protein drugs, e.g., having non-native sequences that can provoke an unwanted immune response. Examples of tolerogenic antigens that are engineered therapeutic proteins not naturally found in humans including human proteins with engineered mutations, e.g., mutations to improve pharmacological characteristics. Examples of tolerogenic antigens that have nonhuman glycosylation include proteins produced in yeast or insect cells.

Tolerogenic antigens can be from proteins that are administered to humans that are deficient in the protein. Deficient means that the patient receiving the protein does not naturally produce enough of the protein. Moreover, the proteins may be proteins for which a patient is genetically deficient. Such proteins include, for example, antithrombin-III, protein C, factor VIII, factor IX, growth hormone, somatotropin, insulin, pramlintide acetate, mecasermin (IGF-1), β-gluco cerebrosidase, alglucosidase-.alpha., laronidase (α-L-iduronidase), idursuphase (iduronate-2-sulphatase), galsulphase, agalsidase-.beta. (α-galactosidase), α-1 proteinase inhibitor, and albumin.

The tolerogenic antigen can be from therapeutic antibodies and antibody-like molecules, including antibody fragments and fusion proteins with antibodies and antibody fragments. These include nonhuman (such as mouse) antibodies, chimeric antibodies, and humanized antibodies. Immune responses to even humanized antibodies have been observed in humans (Getts D R, Getts M T, McCarthy D P, Chastain E M L, & Miller S D (2010), mAbs, 2(6):682-694).

The tolerogenic antigen can be from proteins that are nonhuman. Examples of such proteins include adenosine deaminase, pancreatic lipase, pancreatic amylase, lactase, botulinum toxin type A, botulinum toxin type B, collagenase, hyaluronidase, papain, L-Asparaginase, rasburicase, lepirudin, streptokinase, anistreplase (anisoylated plasminogen streptokinase activator complex), antithymocyte globulin, crotalidae polyvalent immune Fab, digoxin immune serum Fab, L-arginase, and L-methionase.

Tolerogenic antigens include those from human allograft transplantation antigens. Examples of these antigens are the subunits of the various MHC class I and MHC class II haplotype proteins, and single-amino-acid polymorphisms on minor blood group antigens including RhCE, Kell, Kidd, Duffy and Ss.

The tolerogenic antigen can be a self-antigen against which a patient has developed an autoimmune response or may develop an autoimmune response. Examples are proinsulin (diabetes), collagens (rheumatoid arthritis), myelin basic protein (multiple sclerosis). For instance, Type 1 diabetes mellitus (T1D) is an autoimmune disease whereby T cells that recognize islet proteins have broken free of immune regulation and signal the immune system to destroy pancreatic tissue. Numerous protein antigens that are targets of such diabetogenic T cells have been discovered, including insulin, GAD65, chromogranin-A, among others. In the treatment or prevention of T1D, it would be useful to induce antigen-specific immune tolerance towards defined diabetogenic antigens to functionally inactivate or delete the diabetogenic T cell clones.

Tolerance and/or delay of onset or progression of autoimmune diseases may be achieved for various of the many proteins that are human autoimmune proteins, a term referring to various autoimmune diseases wherein the protein or proteins causing the disease are known or can be established by routine testing. In some embodiments, a patient is tested to identify an autoimmune protein and an antigen is created for use in a molecular fusion to create immunotolerance to the protein.

Embodiments can include an antigen, or choosing an antigen from or derived from, one or more of the following proteins. In type 1 diabetes mellitus, several main antigens have been identified: insulin, proinsulin, preproinsulin, glutamic acid decarboxylase-65 (GAD-65), GAD-67, insulinoma-associated protein 2 (IA-2), and insulinoma-associated protein 2.beta. (IA-213); other antigens include ICA69, ICA12 (SOX-13), carboxypeptidase H, Imogen 38, GLIMA 38, chromogranin-A, FISP-60, caboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5. In autoimmune diseases of the thyroid, including Hashimoto's thyroiditis and Graves' disease, main antigens include thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, a main antigen is calcium sensitive receptor. In Addison's disease, main antigens include 21-hydroxylase, 17α-hydroxylase, and P450 side chain cleavage enzyme (P450scc); other antigens include ACTH receptor, P450c21 and P450c17. In premature ovarian failure, main antigens include FSH receptor and .alpha.-enolase. In autoimmune hypophysitis, or pituitary autoimmune disease, main antigens include pituitary gland-specific protein factor (PGSF) 1a and 2; another antigen is type 2 iodothyronine deiodinase. In multiple sclerosis, main antigens include myelin basic protein, myelin oligodendrocyte glycoprotein and proteolipid protein. In rheumatoid arthritis, a main antigen is collagen II. In immunogastritis, a main antigen is H+, K+− ATPase. In pernicious angemis, a main antigen is intrinsic factor. In celiac disease, main antigens are tissue transglutaminase and gliadin. In vitiligo, a main antigen is tyrosinase, and tyrosinase related protein 1 and 2. In myasthenia gravis, a main antigen is acetylcholine receptor. In pemphigus vulgaris and variants, main antigens are desmoglein 3, 1 and 4; other antigens include pemphaxin, desmocollins, plakoglobin, perplakin, desmoplakins, and acetylcholine receptor. In bullous pemphigoid, main antigens include BP180 and BP230; other antigens include plectin and laminin 5. In dermatitis herpetiformis Duhring, main antigens include endomysium and tissue transglutaminase. In epidermolysis bullosa acquisita, a main antigen is collagen VII. In systemic sclerosis, main antigens include matrix metalloproteinase 1 and 3, the collagen-specific molecular chaperone heat-shock protein 47, fibrillin-1, and PDGF receptor; other antigens include Scl-70, U1 RNP, Th/To, Ku, Jol, NAG-2, centromere proteins, topoisomerase I, nucleolar proteins, RNA polymerase I, II and III, PM-Slc, fibrillarin, and B23. In mixed connective tissue disease, a main antigen is UlsnRNP. In Sjogren's syndrome, the main antigens are nuclear antigens SS-A and SS-B; other antigens include fodrin, poly(ADP-ribose) polymerase and topoisomerase. In systemic lupus erythematosus, main antigens include nuclear proteins including SS-A, high mobility group box 1 (HMGB1), nucleosomes, histone proteins and double-stranded DNA. In Goodpasture's syndrome, main antigens include glomerular basement membrane proteins including collagen IV. In rheumatic heart disease, a main antigen is cardiac myosin. Other autoantigens revealed in autoimmune polyglandular syndrome type 1 include aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega.

In some cases, the tolerogenic antigen is a foreign antigen against which a patient has developed an unwanted immune response. Examples are food antigens. Some embodiments include testing a patient to identify foreign antigen and creating a molecular fusion that comprises the antigen and treating the patient to develop immunotolerance to the antigen or food. Examples of such foods and/or antigens are provided. Examples are from peanut: conarachin (Ara h 1), allergen II (Ara h 2), arachis agglutinin, conglutin (Ara h 6); from apple: 31 kda major allergen/disease resistance protein homolog (Mal d 2), lipid transfer protein precursor (Mal d 3), major allergen Mal d 1.03D (Mal d 1); from milk: .alpha.-lactalbumin (ALA), lactotransferrin; from kiwi: actinidin (Act c 1, Act d 1), phytocystatin, thaumatin-like protein (Act d 2), kiwellin (Act d 5); from mustard: 2S albumin (Sin a 1), 11 S globulin (Sin a 2), lipid transfer protein (Sin a 3), profilin (Sin a 4); from celery: profilin (Api g 4), high molecular weight glycoprotein (Api g 5); from shrimp: Pen a 1 allergen (Pen a 1), allergen Pen m 2 (Pen in 2), tropomyosin fast isoform; from wheat and/or other cereals: high molecular weight glutenin, low molecular weight glutenin, alpha- and gamma-gliadin, hordein, secalin, avenin; from strawberry: major strawberry allergy Fra a 1-E (Fra a 1), from banana: profilin (Mus xp 1).

Many protein drugs that are used in human and veterinary medicine induce immune responses, which create risks for the patient and limits the efficacy of the drug. This can occur with human proteins that have been engineered, with human proteins used in patients with congenital deficiencies in production of that protein, and with nonhuman proteins. It would be advantageous to tolerize a recipient to these protein drugs prior to initial administration, and it would be advantageous to tolerize a recipient to these protein drugs after initial administration and development of immune response. In patients with autoimmunity, the self-antigen(s) to which autoimmunity is developed are known. In these cases, it would be advantageous to tolerize subjects at risk prior to development of autoimmunity, and it would be advantageous to tolerize subjects at the time of or after development of biomolecular indicators of incipient autoimmunity. For example, in Type 1 diabetes mellitus, immunological indicators of autoimmunity are present before broad destruction of beta cells in the pancreas and onset of clinical disease involved in glucose homeostasis. It would be advantageous to tolerize a subject after detection of these immunological indicators prior to onset of clinical disease.

g. Neoantigens and Personalized Medicine

In some embodiments the antigen is a neoantigen or a patient-specific antigen. Recent technological improvements have made it possible to identify the immune response to patient-specific neoantigens that arise as a consequence of tumor-specific mutations, and emerging data indicate that recognition of such neoantigens is a maj or factor in the activity of clinical immunotherapies (Schumacher and Schreidber, Science, 348(6230):69-74 (2015). Neoantigen load provides an avenue to selectively enhance T cell reactivity against this class of antigens.

Traditionally, cancer vaccines have targeted tumor-associated antigens (TAAs) which can be expressed not only on tumor cells but in the normal tissues (Ito, et al., Cancer Neoantigens: A Promising Source of Immunogens for Cancer Immunotherapy. J Clin Cell Immunol, 6:322 (2015) doi: 10.4172/2155-9899.1000322). TAAs include cancer-testis antigens and differentiation antigens, and even though self-antigens have the benefit of being useful for diverse patients, expanded T cells with the high-affinity TCR (T-cell receptor) needed to overcome the central and peripheral tolerance of the host, which would impair anti-tumor T-cell activities and increase risks of autoimmune reactions.

Thus, in some embodiments, the antigen is recognized as “non-self” by the host immune system, and preferably can bypass central tolerance in the thymus. Examples include pathogen-associated antigens, mutated growth factor receptor, mutated K-ras, or idiotype-derived antigens. Somatic mutations in tumor genes, which usually accumulate tens to hundreds of fold during neoplastic transformation, could occur in protein-coding regions. Whether missense or frameshift, every mutation has the potential to generate tumor-specific antigens. These mutant antigens can be referred to as “cancer neoantigens” Ito, et al., Cancer Neoantigens: A Promising Source of Immunogens for Cancer Immunotherapy. J Clin Cell Immunol, 6:322 (2015) doi: 10.4172/2155-9899.1000322. Neoantigen-based cancer vaccines have the potential to induce more robust and specific anti-tumor T-cell responses compared with conventional shared-antigen-targeted vaccines. Recent developments in genomics and bioinformatics, including massively parallel sequencing (MPS) and epitope prediction algorithms, have provided a major breakthrough in identifying and selecting neoantigens.

Methods of identifying, selecting, and validating neoantigens are known in the art. See, for example, Ito, et al., Cancer Neoantigens: A Promising Source of Immunogens for Cancer Immunotherapy. J Clin Cell Immunol, 6:322 (2015) doi:10.4172/2155-9899.1000322, which is specifically incorporated by reference herein in its entirety. For example, as discussed in Ito, et al., a non-limiting example of identifying a neoantigen can include screening, selection, and optionally validation of candidate immunogens. First, the whole genome/exome sequence profile is screened to identify tumor-specific somatic mutations (cancer neoantigens) by MPS of tumor and normal tissues, respectively. Second, computational algorithms are used for predicting the affinity of the mutation-derived peptides with the patient's own HLA and/or TCR. The mutation-derived peptides can serve as antigens for the compositions and methods disclosed herein. Third, synthetic mutated peptides and wild-type peptides can be used to validate the immunogenicity and specificity of the identified antigens by in vitro T-cell assay or in vivo immunization. Antigens can be applied to immunotherapy as either biomarkers of responses to immunotherapy, or targets of cancer immunotherapy, including cancer vaccines and adoptive T-cell therapy.

2. Chaperones

The immunizing protein includes a protein chaperone. The protein chaperone increases lymph node uptake; improves immune responses by, for example, increasing the number or ratio of antigen reactive T cells (e.g., activated CD8+ cells); increasing T cell expression of pro-inflammatory molecules including such cytokines, metelloproteases and other molecules including, but not limited to IL-13, TNF-α, IFN-γ, IL-17, IL-6, IL-23, IL-22, IL-21, and MMPs; increases half-life; reduces degradation; or combinations thereof of the peptide chaperone-antigen conjugate or fusion relative to the peptide antigen alone. In some embodiments, the increase in uptake is in lymph node(s) near the site of administration. The lymph node(s) can be draining lymph nodes. In the Examples below, chaperone-antigen was subcutaneously injected in the tail base of mice and elevated presence of the chaperone-antigen relative to antigen alone was found in the draining inguinal lymph nodes.

The Examples below show that the protein chaperones can reduce or prevent a loss of potency of the antigen in the presence of the serum. Thus in some embodiments, the protein chaperone is a protein that when fused to an antigen of interest induces a stronger immune response in vivo than free antigen, even after incubation (e.g., overnight) with serum (e.g., 10% serum).

In some embodiments, the protein chaperone can protect the antigen from degradation in vivo (e.g., from proteases), thus increasing its half-life.

In some embodiments, the protein chaperones are either of sufficiently large molecular weight to facilitate effective lymph node uptake, are binders to endogenous molecules of sufficiently large molecular weight, or a combination thereof. Typically, the size of the chaperone-antigen conjugate or fusion protein is at least about 45 kDa, 50 kDa, 70 kDa, or larger. In some embodiments, the size of the chaperone component alone is at least about 45 kDa, 50 kDa, 70 kDa, or larger. For example, the chaperone can be about 5 kDa and about 500 kDa, or between about 10 Da and about 250 kDa, or between about 25 kDa and about 250 kDa, or between about 40 kDa and about 200 kDa, or about 50 kDa and about 100 kDa, or between about 40 kDa and about 75 kDa.

Preferably the protein chaperone is one that will not induce a systemic immune response in the subject.

Preferred protein chaperones include serum proteins and functional fragments and variants thereof, and other proteins and peptides that bind thereto. Serum proteins include, but are not limited to, albumins, globulins, fibrinogen, regulatory proteins, and clotting factors. Specific examples include Human Serum Albumin, Prealbumin (transthyretin), Alpha 1 antitrypsin, Alpha 1 acid glycoprotein, Alpha 1 fetoprotein, alpha2-macroglobulin, Gamma globulins, Beta-2 microglobulin, Haptoglobin, Ceruloplasmin, Complement component 3, Complement component 4, C-reactive protein (CRP), Lipoproteins (chylomicrons, VLDL, LDL, HDL), Transferrin, Prothrombin, mannose-binding lectin (MBL), and mannose-binding protein (MBP).

In some embodiments, the protein chaperone is a serum protein that is endogenous to the subject. Thus, if the subject to be treated is a human, the protein chaperone can be a human serum protein (e.g., albumins, globulins, fibrinogen, regulatory proteins, clotting factors, etc.), or a functional fragment and variant thereof, and another human protein or peptide that binds thereto.

In some embodiments, the protein chaperone is an Fc fragment. Fc refers to the fragment of an immunoglobulin molecule composed of the constant regions of the heavy chains and responsible for binding to antibody receptors (Fc receptor) on cells and the Clq component of complement. In some embodiments the Fc is human Fc. Suitable Fc fragments include the mouse Fc fragments exemplified below and human homologs and paralogs thereof.

Preferred examples of chaperones include, but are not limited to, albumin, Fc, transthyretin (TTR), and proteins or peptides that bind thereto.

a. Experimental Protein Chaperones

The vaccines exemplified in the experiments below utilized seven different protein carriers: mouse TTR, mouse serum albumin (MSA), the wild-type Fc portion of mouse IgG2a (FcWT), a mutant form of the Fc portion of mouse IgG2a (G236R and L328R) that abrogates interaction with Fcγ receptors (FcKO), and three variants of an archae-derived DNA-binding protein named sso7d. Via mutagenesis, a charge-reduced sso7d variant named rcSso7d was developed. A yeast surface display library was generated using rcSso7d as a scaffold, and two MSA binders were isolated, named M11.1.2 and M18.2.5.

A sequence for TTR is

TTR

(SEQ ID NO: 22)

[symbol]

[symbol]

AGAGESKCPLMVKVLDAVRGSPAV
DVAVKVFKKTSEGSWEPFASGKTAESGELHGLTTDEKFVEGVYRVE
LDTKSYWKTLGISPFHEFADVVFTANDSGHRHYTIAALLSPYSYSTTA
VVSNPQN (UniProtKB - P07309 (TTHY_MOUSE)),

wherein the leader sequence is in bold and italics.

SEQ ID NO:24, without the leader sequence is

(SEQ ID NO: 26)

GPAGAGESKCPLMVKVLDAVRGSPAVDVAVKVFKKTSEGSWEPFASGKTA
ESGELHGLTTDEKFVEGVYRVELDTKSYWKTLGISPFHEFADVVFTANDS
GHRHYTIAALLSPYSYSTTAVVSNPQN.
MSA

(SEQ ID NO: 6)

EAHKSEIAHRYNDLGEQHFKGLVLIAFSQYLQKCSYDEHAKLVQEVTDFA
KTCVADESAANCDKSLHTLFGDKLCAIPNLRENYGELADCCTKQEPERNE
CFLQHKDDNPSLPPFERPEAEAMCTSFKENPTTFMGHYLHEVARRHPYFY
APELLYYAEQYNEILTQCCAEADKESCLTPKLDGVKEKALVSSVRQRMKC
SSMQKFGERAFKAWAVARLSQTFPNADFAEITKLATDLTKVNKECCHGDL
LECADDRAELAKYMCENQATISSKLQTCCDKPLLKKAHCLSEVEHDTMPA
DLPAIAADFVEDQEVCKNYAEAKDVFLGTFLYEYSRRHPDYSVSLLLRLA
KKYEATLEKCCAEANPPACYGTVLAEFQPLVEEPKNLVKTNCDLYEKLGE
YGFQNAILVRYTQKAPQVSTPTLVEAARNLGRVGTKCCTLPEDQRLPCVE
DYLSAILNRVCLLHEKTPVSEHVTKCCSGSLVERRPCFSALTVDETYVPK
EFKAETFTFHSDICTLPEKEKQIKKQTALAELVKHKPKATAEQLKTVMDD
FAQFLDTCCKAADKDTCFSTEGPNLVTRCKDALA (NCBI: P07724
(precursor to MSA))
FcWT

(SEQ ID NO: 7)

EPRVPITQNPCPPLKECPPCAAPDLLGGPSVFIFPPKIKDVLMISLSPMV
TCVVVDVSEDDPDVQISWFVNNVEVHTAQTQTHREDYNSTLRVVSALPIQ
HQDWMSGKEFKCKVNNRALPSPIEKTISKPRGPVRAPQVYVLPPPAEEMT
KKEFSLTCMITGFLPAEIAVDWTSNGRTEQNYKNTATVLDSDGSYFMYSK
LRVQKSTWERGSLFACSVVHEGLHNHLTTKTISRSLGK
FcKO

(SEQ ID NO: 8)

EPRVPITQNPCPPLKECPPCAAPDLLRGPSVFIFPPKIKDVLMISLSPMV
TCVVVDVSEDDPDVQISWFVNNVEVHTAQTQTHREDYNSTLRVVSALPIQ
HQDWMSGKEFKCKVNNRARPSPIEKTISKPRGPVRAPQVYVLPPPAEEMT
KKEFSLTCMITGFLPAEIAVDWTSNGRTEQNYKNTATVLDSDGSYFMYSK
LRVQKSTWERGSLFACSVVHEGLHNHLTTKTISRSLGK
M11.1.2

(SEQ ID NO: 9)

ATVKYTYRGEEKRVDISKIKWVNRWGQHLAFKYDKGGGAAGYGWVSEKDA
PKELLQMLEKR
M18.2.5

(SEQ ID NO: 10)

ATVKFTYRGEEKQVDISKIKWVVRLGQVIMFKYDEGGGASGYGRVSEKDA
PKELLQMLEK
rcSso7d

(SEQ ID NO: 11)

ATVKFTYQGEEKQVDISKIKKVWRVGQMISFTYDEGGGATGRGAVSEKDA
PKELLQMLEKQ

b. Exemplary Human Protein Chaperones

In some preferred embodiments, the chaperone protein is endogenous to the subject to which the fusion protein will be administered. Thus, in some embodiments when the fusion protein will be administered to a human subject, the chaperone protein is a human protein or a fragment or variant thereof. Exemplary human protein chaperones include, for example:

A sequence for human TTR is

Human TTR

(SEQ ID NO: 23)

[symbol]

[symbol]

GPTGTGESKCPLMVKVLDAVRGSPAIN
VAVHVFRKAADDTWEPFASGKTSESGELHGLTTEEEFVEGIYKVEID
TKSYWKALGISPFHEH AEVVFTANDS GPRRYTIAAL LSPYSYSTTA
VVTNPKE (UniProtKB - P02766 (TTHY_HUMAN),

wherein the leader sequence is in bold and italics.

SEQ ID NO:23, without the leader sequence is

(SEQ ID NO: 29)

GPTGTGESKCPLMVKVLDAVRGSPAINVAVHVFRKAADDTWEPFASGKTS
ESGELHGLTTEEEFVEGIYKVEIDTKSYWKALGISPFHEHAEVVFTANDS
GPRRYTIAALLSPYSYSTTAVVTNPKE.

Functional variants of Human TTR are well known in the art and include those discussed in UniProtKB—P02766 (TTHY_HUMAN), which is specifically incorporated by reference herein in its entirety.

TTR is a particularly attractive chaperone because it forms a homotetramer, thus providing four copies of antigen cargo per protein. As shown in the Examples below, this can allow for use of a lower dosage of protein relative to other chaperones. Because TTR is a tetramer, its use as an antigen carrier opens up the possibility of co-delivering multiple antigens (up to four) at once. For example, two, three, or four different TTR-antigen fusion proteins can be mixed together to form heterotetramers.

Human Serum Albumin

(SEQ ID NO: 24)

[symbol]

[symbol]

[symbol]

AHKSEVAHRFKDLGEENFKALV
LIAFAQYLQQCPFEDHVKLVNEVTEFAKTCVADESAENCDKSLHTLFGDK
LCTVATLRETYGEMADCCAKQEPERNECFLQHKDDNPNLPRLVRPEVDVM
CTAFHDNEETFLKKYLYEIARRHPYFYAPELLFFAKRYKAAFTECCQAAD
KAACLLPKLDELRDEGKASSAKQRLKCASLQKFGERAFKAWAVARLSQRF
PKAEFAEVSKLVTDLTKVHTECCHGDLLECADDRADLAKYICENQDSISS
KLKECCEKPLLEKSHCIAEVENDEMPADLPSLAADFVESKDVCKNYAEAK
DVFLGMFLYEYARRHPDYSVVLLLRLAKTYETTLEKCCAAADPHECYAKV
FDEFKPLVEEPQNLIKQNCELFEQLGEYKFQNALLVRYTKKVPQVSTPTL
VEVSRNLGKVGSKCCKHPEAKRMPCAEDYLSVVLNQLCVLHEKTPVSDRV
TKCCTESLVNRRPCFSALEVDETYVPKEFNAETFTFHADICTLSEKERQI
KKQTALVELVKHKPKATKEQLKAVMDDFAAFVEKCCKADDKETCFAEEGK
KLVAASQAALGL (UniProtKB - P02768 (ALBU_HUMAN)),

wherein the leader sequence is in bold and italics.

SEQ ID NO:24, without the leader sequence is

(SEQ ID NO: 28)

DAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNE
CFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKC
ASLQKFGERAFKAWAVARLSQRFPKAEFAEVSKLVTDLTKVHTECCHGDL
LECADDRADLAKYICENQDSISSKLKECCEKPLLEKSHCIAEVENDEMPA
DLPSLAADFVESKDVCKNYAEAKDVFLGMFLYEYARRHPDYSVVLLLRLA
KTYETTLEKCCAAADPHECYAKVFDEFKPLVEEPQNLIKQNCELFEQLGE
YKFQNALLVRYTKKVPQVSTPTLVEVSRNLGKVGSKCCKHPEAKRMPCAE
DYLSVVLNQLCVLHEKTPVSDRVTKCCTESLVNRRPCFSALEVDETYVPK
EFNAETFTFHADICTLSEKERQIKKQTALVELVKHKPKATKEQLKAVMDD
FAAFVEKCCKADDKETCFAEEGKKLVAASQAALGL

Functional variants of human serum albumin are well known in the art and include those discussed in UniProtKB—P02768 (ALBU_HUMAN), which is specifically incorporated by reference herein in its entirety.

c. Functional Fragments and Variants

Functional fragments and variants of the disclosed proteins sequences, sso7D, and other suitable protein chaperones including the serum proteins discussed herein are also provided. Functional fragments and variants can be, for example, sufficiently large molecular weight (e.g., about 45 kDa or greater) to facilitate effective lymph node uptake, bind to endogenous molecules of sufficiently large molecular weight, or a combination thereof. Typically the functional fragment is a fragment sufficient to increase lymph node uptake; improve immune responses by, for example, increasing the number or ratio of antigen reactive T cells (e.g., activated CD8+ cells); increasing T cell expression of pro-inflammatory molecules including such cytokines, metelloproteases and other molecules including, but not limited to IL-1β, TNF-α, IFN-γ, IL-17, IL-6, IL-23, IL-22, IL-21, and MMPs; increases half-life; reduces degradation; or combinations thereof of the peptide chaperone-antigen conjugate or fusion relative to the peptide antigen alone.

In some embodiments, a fragment or variant has 1, 2, 3, 4, 5, 6, 7, 8, 9, 10, 15, 20, 25, 30, 35, or more amino acids deleted from the N-terminus, the C-terminus, or both relative to the full-length protein. For example, proteins missing the N-terminal methionine or the entire endogenous signal peptide, are expressly disclosed. A signal peptide (also referred to as signal sequence, targeting signal, localization signal, localization sequence, transit peptide, leader sequence, and leader peptide) is a short (5-30 amino acids long) peptide present at the N-terminus of the majority of newly synthesized proteins that are destined towards the secretory pathway.

In some embodiments, a fusion protein expression construct includes an endogenous or heterologous leader sequence which can be cleaved when expressed by cells. In some embodiments, the fusion protein does not include a leader sequence. Thus all the sequences and proteins disclosed herein, and fusion proteins derived therefrom, are expressly disclosed both with and without a leader sequence. In some embodiments, a fusion protein with or without a leader sequences can be administered to a subject.

In some embodiments, the endogenous leader sequence of chaperone protein is replaced with an alternative leader sequence. Thus, in some embodiments, the leader sequence is heterologous to the chaperone protein. A non-limiting example is MRVPAQLLGLLLLWLPGARCA (SEQ ID NO:25).

In some embodiments, variants have one or more insertions, deletions, substitutions, or other selected modifications of particular regions or specific amino acids residues, provided the activity of the protein is not significantly altered or impaired compared to the non-modified protein.

For example, the chaperone can be a variant or functional fragment of any of disclosed proteins having at least 50, 60, 70, 75, 80, 85, 90, 95, 96, 97, 98, 99 percent sequence identity to the disclosed protein.

In some embodiments, the size of the chaperone-antigen conjugate or fusion protein is at least about 45 kDa, 50 kDa, or larger. In some embodiments, the size of the chaperone component alone is at least about 45 kDa, 50 kDa, or larger.

3. Linkers

The disclosed fusion proteins optionally contain one or more peptide or other linker domains. The linkers can be used to operably link or connect two domains, regions, or sequences of the fusion protein. In some embodiments, one or more linkers separate the peptide antigen from the protein chaperone.

Peptide linker sequences are typically at least 2 amino acids in length. Preferably the peptide domains are flexible peptides or polypeptides. A “flexible linker” herein refers to a peptide or polypeptide containing two or more amino acid residues joined by peptide bond(s) that provides increased rotational freedom for two polypeptides linked thereby than the two linked polypeptides would have in the absence of the flexible linker. Such rotational freedom allows two or more antigen binding sites joined by the flexible linker to each access target antigen(s) more efficiently. Exemplary flexible peptides/polypeptides include, but are not limited to, the amino acid sequences Gly-Ser, Gly-Ser-Gly-Ser (SEQ ID NO:12), Ala-Ser, Gly-Gly-Gly-Ser (SEQ ID NO:13), (Gly₄-Ser)₃ (SEQ ID NO: 14), (Gly₄-Ser)₄ (SEQ ID NO:15), GGGSHHHHHHGGGS (SEQ ID NO:27). In SEQ ID NO:27, GGGS (SEQ ID NO: 13) can serve as a flexible linker and HHHHHH (SEQ ID NO:30) can serve as an aid in purification (e.g., His-Tag), as an additional spacer, or a combination thereof. Additional flexible peptide/polypeptide sequences are well known in the art.

In some embodiments, the linker includes a glycine-glutamic acid di-amino acid sequence.

In some embodiments, the fusion protein includes two or more linkers.

In some embodiments, the linker domain is or includes another peptide or protein domain, for example, a purification tag between two flexible linkers.

In some embodiments, the linker increases or enhances the ability of the fusion protein or conjugate to (1) remain in the tissue, (2) avoid systemic circulation, (3) accumulate in the lymph node, or (4) a combination thereof relative to the same chaperone-antigen in the absence of the linker.

Non-limiting exemplary chaperone-antigen fusion proteins including linkers can have the structure:

Chaperone Protein

(SEQ ID NO: 27)

GGGSHHHHHHGGGS Antigenic peptide

Non-limiting exemplary chaperone-antigen fusion proteins including linkers for delivery to two antigens can have the structure:

Chaperone Protein

(SEQ ID NO: 27)

GGGSHHHHHHGGGS
Antigenic Peptide 1

(SEQ ID NO: 13)

GGGS Antigenic Peptide 2

4. Targeting Moieties

In some embodiments the composition (e.g., fusion protein, conjugate, or carrier thereof) is modified to include one or more targeting signals or domains. The targeting signal can include a sequence of monomers that facilitates in vivo localization of the molecule. The monomers can be amino acids, nucleotide or nucleoside bases, or sugar groups such as glucose, galactose, and the like which form carbohydrate targeting signals. Targeting signals or sequences can be specific for a host, tissue, organ, cell, organelle, non-nuclear organelle, or cellular compartment. For example, in some embodiments the composition includes both a cell-specific targeting domain and an organelle specific targeting domain to enhance delivery of the polypeptide to a subcellular organelle of a specific cells type.

a. Cell Targeting

The proteins of interest disclosed herein can be modified to target a specific cell type or population of cells.

For example, the targeting signal can bind to its ligand or receptor which is located on the surface of a target cell such as to bring the composition and cell membranes sufficiently close to each other to allow penetration of the composition into the cell.

In a preferred embodiment, the targeting molecule is selected from the group consisting of an antibody or antigen binding fragment thereof, an antibody domain, an antigen, a dendritic cell receptor, a cell surface receptor, a cell surface adhesion molecule, a major histocompatibility locus protein, a viral envelope protein and a peptide selected by phage display that binds specifically to a defined cell.

Targeting a composition of interest to specific cells can be accomplished by modifying the composition of interest to express specific cell and tissue targeting signals. These sequences target specific cells and tissues. In some embodiments the interaction of the targeting signal with the cell does or does not occur through a traditional receptor:ligand interaction. The eukaryotic cell comprises a number of distinct cell surface molecules. The structure and function of each molecule can be specific to the origin, expression, character and structure of the cell. Determining the unique cell surface complement of molecules of a specific cell type can be determined using techniques well known in the art.

One skilled in the art will appreciate that the tropism of the proteins of interest described can be altered by changing the targeting signal. In one specific embodiment, cell surface antigen specific antibodies are used to the composition.

It is known in the art that nearly every cell type in a tissue in a mammalian organism possesses some unique cell surface receptor or antigen. Thus, it is possible to incorporate nearly any ligand for the cell surface receptor or antigen as a targeting signal. For example, peptidyl hormones can be used a targeting moieties to target delivery to those cells which possess receptors for such hormones. Chemokines and cytokines can similarly be employed as targeting signals to target delivery of the complex to their target cells. A variety of technologies have been developed to identify genes that are preferentially expressed in certain cells or cell states and one of skill in the art can employ such technology to identify targeting signals which are preferentially or uniquely expressed on the target tissue of interest

In particular preferred embodiments, the targeting signal targets an antigen presenting cell. Antigen-presenting cells (APCs) are a heterogeneous group of immune cells that mediate the cellular immune response by processing and presenting antigens for recognition by certain lymphocytes such as T cells. Classical APCs include dendritic cells (DC), macrophages, Langerhans cells and B cells.

For example, cross-presenting dendritic cells are known to express an internalizing receptor named DEC-205. Targeting antigen to DEC-205 has been shown to facilitate effective cross presentation and T cell activation. An example of DEC-205 binding fibronectin is DEC1, and its use as a targeting moiety is illustrated in the Examples below (see, e.g., FIG. 7). Thus DC targeting agents can be added to fusion protein vaccines.

In addition to DEC-205, other potential DC targets include, but are not limited to, mannose receptor, mannose binding lectin, ficolins, DC-SIGN, DCAR, DCIR, dectins, DLEC, scavenger receptors, F4/80, Fc receptor, and DC-STAMP.

Exemplary targeting agents include antibodies, scFv and other non-antibody scaffold proteins including fibronectin, sso7d, knottin, DARPin, etc.

b. Antibodies

In some embodiments, the targeting moiety is an antibody or antigen binding fragment thereof bound to the disclosed composition and acting as the targeting signal. The antibodies or antigen binding fragment thereof are useful for directing the composition to a cell type or cell state. In some embodiments, the polypeptide of interest possesses an antibody binding domain, for example from proteins known to bind antibodies such as Protein A and Protein G from Staphylococcus aureus.

Other domains known to bind antibodies are known in the art and can be substituted. In certain embodiments, the antibody is polyclonal, monoclonal, linear, humanized, chimeric or a fragment thereof. Representative antibody fragments are those fragments that bind the antibody binding portion of the non-viral vector and include Fab, Fab′, F(ab′), Fv diabodies, linear antibodies, single chain antibodies and bispecific antibodies known in the art.

In some embodiments, the targeting domain includes all or part of an antibody that directs the composition to the desired target cell type or cell state. Antibodies can be monoclonal or polyclonal, but are preferably monoclonal. Antibodies can be derived from human genes, specific for cell surface markers, and produced to reduce potential immunogenicity to a human host as is known in the art. For example, transgenic mice which contain the entire human immunoglobulin gene cluster are capable of producing “human” antibodies can be utilized. In one embodiment, fragments of such human antibodies are employed as targeting signals. In a preferred embodiment, single chain antibodies modeled on human antibodies are prepared in prokaryotic culture.

5. Additional Sequences

The fusion protein can optionally include additional sequences or moieties, including, but not limited to linkers and purification tags.

In a preferred embodiment the purification tag is a polypeptide. Polypeptide purification tags are known in the art and include, but are not limited to, HIS tags which typically include six or more, typically consecutive, histidine residues; FLAG tags, which typically include the sequence DYKDDDDK (SEQ ID NO: 16); haemagglutinin (HA) for example, YPYDVP (SEQ ID NO: 17); MYC tag for example ILKKATAYIL (SEQ ID NO:18) or EQKLISEEDL (SEQ ID NO:19). Methods of using purification tags to facilitate protein purification are known in the art and include, for example, a chromatography step wherein the tag reversibly binds to a chromatography resin.

Purifications tags can be N-terminal or C-terminal to the fusion protein, or can between a central domain between the peptide antigen and the protein chaperone domains. The purification tags N-terminal to the fusion protein are typically separated from the polypeptide of interest at the time of the cleavage in vivo. Therefore, purification tags N-terminal to the fusion protein can be used to remove the fusion protein from a cellular lysate following expression and extraction of the expression or solubility enhancing amino acid sequence, but cannot be used to remove the polypeptide of interest. Purification tags C-terminal to the fusion protein can be used to remove the polypeptide of interest from a cellular lysate following expression of the fusion protein, but cannot be used to remove the expression or solubility enhancing amino acid sequence. Purification tags that are C-terminal to the expression or solubility enhancing amino acid sequence can be N-terminal to, C-terminal to, or incorporated within the sequence of the polypeptide of interest.

B. Adjuvants

The peptide antigens can be administered alone, or in combination with an adjuvant. The adjuvants exemplified in experiments described below include a lipidated CpG molecule previously described (Liu, et al., Nature Letters, 507:519-22 (+11 pages of extended data) (2014)), unformulated CpG, polyinosinic:polycytidylic acid (polyIC), and cyclic dinucleotides (CDN).

1. Immunostimulatory Oligonucleotides

The adjuvants can be an immunostimulatory oligonucleotide. In some embodiments, the immunostimulatory oligonucleotide can serve as a ligand for pattern recognition receptors (PRRs). Examples of PRRs include the Toll-like family of signaling molecules that play a role in the initiation of innate immune responses and also influence the later and more antigen specific adaptive immune responses. Therefore, the oligonucleotide can serve as a ligand for a Toll-like family signaling molecule, such as Toll-Like Receptor 9 (TLR9).

For example, unmethylated CpG sites can be detected by TLR9 on plasmacytoid dendritic cells and B cells in humans (Zaida, et al., Infection andlmmunity, 76(5):2123-2129, (2008)). Therefore, the sequence of oligonucleotide can include one or more unmethylated cytosine-guanine (CG or CpG, used interchangeably) dinucleotide motifs. The ‘p’ refers to the phosphodiester backbone of DNA, as discussed in more detail below, some oligonucleotides including CG can have a modified backbone, for example a phosphorothioate (PS) backbone.

In some embodiments, an immunostimulatory oligonucleotide can contain more than one CG dinucleotide, arranged either contiguously or separated by intervening nucleotide(s). The CpG motif(s) can be in the interior of the oligonucleotide sequence. Numerous nucleotide sequences stimulate TLR9 with variations in the number and location of CG dinucleotide(s), as well as the precise base sequences flanking the CG dimers.

Typically, CG ODNs are classified based on their sequence, secondary structures, and effect on human peripheral blood mononuclear cells (PBMCs). The five classes are Class A (Type D), Class B (Type K), Class C, Class P, and Class S (Vollmer, J & Krieg, A M, Advanced drug delivery reviews 61(3): 195-204 (2009), incorporated herein by reference). CG ODNs can stimulate the production of Type I interferons (e.g., IFNα) and induce the maturation of dendritic cells (DCs). Some classes of ODNs are also strong activators of natural killer (NK) cells through indirect cytokine signaling. Some classes are strong stimulators of human B cell and monocyte maturation (Weiner, GL, PNAS USA 94(20): 10833-7 (1997); Dalpke, AH, Immunology 106(1): 102-12 (2002); Hartmann, G, J of Immun. 164(3):1617-2 (2000), each of which is incorporated herein by reference).

Other PRR Toll-like receptors include TLR3, and TLR7 which may recognize double-stranded RNA, single-stranded and short double-stranded RNAs, respectively, and retinoic acid-inducible gene I (RIG-I)-like receptors, namely RIG-I and melanoma differentiation-associated gene 5 (MDA5), which are best known as RNA-sensing receptors in the cytosol. Therefore, in some embodiments, the oligonucleotide contains a functional ligand for TLR3, TLR7, or RIG-I-like receptors, or combinations thereof.

Examples of immunostimulatory oligonucleotides, and methods of making them are known in the art, see for example, Bodera, P. Recent Pat Inflamm Allergy Drug Discov. 5(1):87-93 (2011), incorporated herein by reference.

In some embodiments, the oligonucleotide includes two or more immunostimulatory sequences.

2. Lipidated Adjuvants

In some embodiments, a lipidated adjuvant such as those described in Liu, et al., Nature Letters, 507:519-22 (+11 pages of extended data) (2014)) (lipo-CpG) and U.S. Pat. No. 9,107,904, each of which is specifically incorporated by reference herein in its entirety. In some embodiments, the lipidated adjuvant includes an immunostimulatory oligonucleotide linked to a lipid. The lipid conjugates typically include a hydrophobic lipid. The lipid can be linear, branched, or cyclic. The lipid is preferably at least 17 to 18 carbons in length, but may be shorter if it shows good albumin binding and adequate targeting to the lymph nodes.

Lymph node-targeting conjugates include lipid-oligonucleotide conjugates and lipid-peptide conjugates that can be trafficked from the site of delivery through the lymph to the lymph node. In preferred embodiments, the activity relies, in-part, on the ability of the conjugate to associate with albumin in the blood of the subject. Therefore, lymph node-targeted conjugates typically include a lipid that can bind to albumin under physiological conditions. Lipids suitable for targeting the lymph node can be selected based on the ability of the lipid or a lipid conjugate including the lipid to bind to albumin.

Examples of preferred lipids for use in lymph node targeting lipid conjugates include, but are not limited to fatty acids with aliphatic tails of 8-30 carbons including, but not limited to, linear and unsaturated and saturated fatty acids, branched saturated and unsaturated fatty acids, and fatty acids derivatives, such as fatty acid esters, fatty acid amides, and fatty acid thioesters, diacyl lipids, Cholesterol, Cholesterol derivatives, and steroid acids such as bile acids; Lipid A or combinations thereof.

In some embodiments, the lipid is a diacyl lipid or two-tailed lipid. Diacyllipids include but not limited to, ester bond linkage, amide bond linkage, thioester bond linkage. In a particular embodiment, the diacyllipids are phosphate lipids, glycolipids, and sphingolipids.

In some embodiments, the tails in the diacyl lipid contain from about 8 to about 30 carbons and can be saturated, unsaturated, or combinations thereof. The tails can be coupled to the head group via ester bond linkages, amide bond linkages, thioester bond linkages, or combinations thereof. In a particular embodiment, the diacyl lipids are phosphate lipids, glycolipids, sphingolipids, or combinations thereof.

Preferably, lymph node-targeting conjugates include a lipid that is 8 or more carbon units in length. It is believed that increasing the number of lipid units can reduce insertion of the lipid into plasma membrane of cells, allowing the lipid conjugate to remain free to bind albumin and traffic to the lymph node.

For example, the lipid can be a diacyl lipid composed of two C18 hydrocarbon tails.

In some embodiments, the lipid for use in preparing lymph node targeting lipid conjugates is not a single chain hydrocarbon (e.g., C18), or cholesterol. Cholesterol conjugation has been explored to enhance the immunomodulation of molecular adjuvants such as CpG and immunogenicity of peptides, but cholesterol conjugates, which associates well with lipoproteins but poorly with albumin, show poor lymph node targeting and low immunogenicity in vaccines compared to optimal albumin-binding conjugates.

3. Other Adjuvants

Other adjuvants are also known in the art and can be used in the disclosed compositions and methods. The adjuvant may be without limitation alum (e.g., aluminum hydroxide, aluminum phosphate); saponins purified from the bark of the Q. saponaria tree such as QS21 (a glycolipid that elutes in the 21st peak with HPLC fractionation; Antigenics, Inc., Worcester, Mass.); poly [di(carboxylatophenoxy)phosphazene] (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic).

Adjuvants may be TLR ligands, such as those discussed above. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) and threonyl-muramyl dipeptide (t-MDP; Ribi); OM-174 (a glucosamine disaccharide related to lipid A; OM Pharma S A, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

The adjuvant can also be oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-gamma), macrophage colony stimulating factor, and tumor necrosis factor.

C. Nucleic Acids

Nucleic acid sequences encoding peptides, proteins, fusion proteins, and adjuvants, and isolated non-coding nucleic acid adjuvants are also provided. Thus the disclosed compositions include embodiments in which the antigenic fusion protein, protein adjuvant, or a combination thereof are administered to the subject as nucleic acid encoding the antigenic fusion protein and protein adjuvant, and which is subsequently expressed by the subject's cells to express the antigenic fusion protein, protein adjuvant, or a combination thereof. Thus in some embodiments, the therapy includes in vivo delivery of nucleic acids. Expression of the fusion protein can be enhanced by delivering the composition as nucleic acid. Nucleic acid delivery can also have the advantage of passing endoplasmic reticulum (ER) quality control and are easy to construct.

For example, in some embodiments, the composition is, or includes, a DNA vaccine. DNA immunization provides a non-replicating transcription unit that serves as a template for the synthesis of proteins or protein segments to induce antigen specific immune responses in the host (Ho, et al., Autoimmunity, 39(8):675-682 (2006)). Injection of DNA encoding foreign antigens can promote immunity against a variety of microbes and tumors. In autoimmune diseases DNA vaccines induce tolerance to the DNA-encoded self-antigens. The DNA-encoded self-antigen depends on the disease to be treated, and can be determined by one of skill in the art.

Additionally or alternatively, nucleic acids encoding the chaperone-antigen can be expressed in cells in vitro, the proteins collected (e.g., by protein purification), and administered to the subject.

1. Isolated Nucleic Acids

Isolated nucleic acids are provided. As used herein, “isolated nucleic acid” refers to a nucleic acid that is separated from other nucleic acid molecules that are present in a mammalian genome, including nucleic acids that normally flank one or both sides of the nucleic acid in a mammalian genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids can be in sense or antisense orientation, or can be complementary to a reference sequence. Reference sequences include, for example, the nucleotide sequence of antigenic fusion protein, protein adjuvant which are discussed above.

Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone.

2. Vectors and Host Cells

Nucleic acids, such as those described above, can be inserted into vectors for expression in cells. As used herein, a “vector” is a replicon, such as a plasmid, phage, or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen Life Technologies (Carlsbad, Calif.).

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the polypeptides described herein.

D. Delivery Vehicles

The compositions can be administered and taken up into the cells of a subject with or without the aid of a delivery vehicle. Any of the delivery vehicles can include a targeting moiety such as those introduced above. Appropriate delivery vehicles for the disclosed compositions are known in the art and can be selected to suit the particular composition. For example, if the composition is a nucleic acid or vector, the delivery vehicle can be a viral vector, for example a commercially available preparation, such as an adenovirus vector (Quantum Biotechnologies, Inc. (Laval, Quebec, Canada). The viral vector delivery can be via a viral system, such as a retroviral vector system which can package a recombinant retroviral genome (see e.g., Pastan et al., (1988) Proc. Natl. Acad. Sci. U.S.A. 85:4486; Miller et al., (1986) Mol. Cell. Biol. 6:2895). The recombinant retrovirus can then be used to infect and thereby deliver to the infected cells nucleic acid encoding the compositions. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948 (1994)), adeno-associated viral (AAV) vectors (Goodman et al., Blood 84:1492-1500 (1994)), lentiviral vectors (Naidini et al., Science 272:263-267 (1996)), pseudotyped retroviral vectors (Agrawal et al., Exper. Hematol. 24:738-747 (1996)).

Physical transduction techniques can also be used, such as liposome delivery and receptor-mediated and other endocytosis mechanisms (see, for example, Schwartzenberger et al., Blood 87:472-478 (1996)). For example in some embodiments, the composition is delivered via a liposome. Commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art are well known.

In addition, the nucleic acids can be delivered in vivo by electroporation, the technology for which is available from Genetronics, Inc. (San Diego, Calif.) as well as by means of a SONOPORATION machine (ImaRx Pharmaceutical Corp., Tucson, Ariz.). This disclosed compositions and methods can be used in conjunction with any of these or other commonly used gene transfer methods.

In some embodiments, the composition is incorporated into or encapsulated by a nanoparticle, microparticle, micelle, synthetic lipoprotein particle, or carbon nanotube. For example, the composition can be incorporated into a vehicle such as polymeric microparticles which provide controlled release of the composition. In some embodiments, release of the composition is controlled by diffusion of the composition out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof.

E. Formulations

1. Pharmaceutical Compositions

Pharmaceutical compositions including a chaperone-antigen, an adjuvant, or combination thereof, or a delivery vehicle including a chaperone-antigen, an adjuvant, or combination thereof are provided. Pharmaceutical compositions can be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

The pharmaceutical compositions can include an effective amount of chaperone-antigen. Effective amounts are discussed in more detail below. As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally dosage levels of 0.001 to 10 mg/kg of body weight of mammals. Administration can be determined by the intended use, and include, for example, daily, every other day, every three days, once a week, once every 10 days, once a month, once every 6 weeks, once every two months, etc. Exemplary vaccine schedules are provided below. Generally, for intravenous injection or infusion, dosage may be lower.

In a preferred embodiment the chaperone-antigens are administered in an aqueous solution, by parenteral injection. In some embodiments, the composition includes albumin, or other serum proteins.

The formulation can be in the form of a suspension or emulsion. In general, pharmaceutical compositions are provided including an effective amount of the chaperone-antigen and optionally include pharmaceutically acceptable diluents, preservatives, solubilizers, emulsifiers, adjuvants and/or carriers. Such compositions can include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Immunogenic Compositions

The antigen-chaperones disclosed herein can be used in immunogenic compositions and as components in vaccines. Typically, immunogenic compositions disclosed herein include an adjuvant, an antigen, or a combination thereof. The combination of an adjuvant and an antigen can be referred to as a vaccine. When administered to a subject in combination, the adjuvant and antigen can be administered in separate pharmaceutical compositions, or they can be administered together in the same pharmaceutical composition.

F. Combination Therapies

In some embodiments, the chaperone-antigen alone or in combination with an adjuvant is administered in combination with one or more additional therapeutic agents. The agents can be administered in the same pharmaceutical composition as the chaperone-antigen alone or in combination with adjuvant, or can be administered in separate pharmaceutical compositions. Therefore, the term “combination” or “combined” is used to refer to either concomitant, simultaneous, or sequential administration of the different agents. The combinations can be administered either concomitantly (e.g., as an admixture), separately but simultaneously (e.g., via separate intravenous lines into the same subject; one agent is given orally while the other agent is given by infusion or injection, etc.,), or sequentially (e.g., one agent is given first followed by the second).

In some embodiments, the chaperone-antigen is administered in combination with a conventional therapeutic agent used for treatment of the disease or condition being treated. Conventional therapeutics agents are known in the art and can be determined by one of skill in the art based on the disease or disorder to be treated. For example, if the disease or condition is cancer, the chaperone-antigen can be co-administered with a chemotherapeutic drug; or if the disease or condition is a bacterial infection, the chaperone-antigen can be co-administered with an antibiotic.

1. Immunostimulatory

In preferred embodiments, the chaperone-antigen alone or with an adjuvant is administered in combination with another immunostimulatory agent. The experiments in the Examples below show that when peptide chaperone-antigens are delivered with an adjuvant (as a vaccine) as part of a combination immunotherapy regimen including (i) a tumor targeting antibody (ii) an extended serum half-life IL-2 (MSA-IL2) (see Zhu, et al., Cancer Cell, 27:489-501 (2015)), which is specifically incorporated by reference herein in its entirety) and (iii) checkpoint inhibitor antibodies like anti-PD-1 and anti-CTLA4, they can elicit potent anti-tumor CD8+ T-cell responses and result in dramatic tumor regression.

Non-limiting examples of tumor-targeting antibodies include clinical antibodies such as Trastuzumab (targeting HER2/neu), Cetuximab (targeting EGFR), Rituximab, (targeting CD20), and antibodies useful for pre-clinical testing, such as TA99 (targeting Trp1 in melanoma models) and sm3E (targeting CEA) which are used in mouse models.

In some embodiments, the compositions are administered in combination with a PD-1 antagonist, CTLA-4 antagonist, or a combination thereof

a. PD-1 Antagonists

Activation of T cells normally depends on an antigen-specific signal following contact of the T cell receptor (TCR) with an antigenic peptide presented via the major histocompatibility complex (MHC) while the extent of this reaction is controlled by positive and negative antigen-independent signals emanating from a variety of co-stimulatory molecules. The latter are commonly members of the CD28/B7 family. Conversely, Programmed Death-1 (PD-1) is a member of the CD28 family of receptors that delivers a negative immune response when induced on T cells. Contact between PD-1 and one of its ligands (B7-H1 or B7-DC) induces an inhibitory response that decreases T cell multiplication and/or the strength and/or duration of a T cell response. Suitable PD-1 antagonists are described in U.S. Pat. Nos. 8,114,845, 8,609,089, and 8,709,416, and include compounds or agents that either bind to and block a ligand of PD-1 to interfere with or inhibit the binding of the ligand to the PD-1 receptor, or bind directly to and block the PD-1 receptor without inducing inhibitory signal transduction through the PD-1 receptor.

In some embodiments, the PD-1 receptor antagonist binds directly to the PD-1 receptor without triggering inhibitory signal transduction and also binds to a ligand of the PD-1 receptor to reduce or inhibit the ligand from triggering signal transduction through the PD-1 receptor. By reducing the number and/or amount of ligands that bind to PD-1 receptor and trigger the transduction of an inhibitory signal, fewer cells are attenuated by the negative signal delivered by PD-1 signal transduction and a more robust immune response can be achieved.

It is believed that PD-1 signaling is driven by binding to a PD-1 ligand (such as B7-H1 or B7-DC) in close proximity to a peptide antigen presented by major histocompatibility complex (MHC) (see, for example, Freeman, Proc. Natl. Acad. Sci. U. S. A, 105: 10275-10276 (2008)).

Therefore, proteins, antibodies or small molecules that prevent co-ligation of PD-1 and TCR on the T cell membrane are also useful PD-1 antagonists.

In preferred embodiments, the PD-1 receptor antagonists are small molecule antagonists or antibodies that reduce or interfere with PD-1 receptor signal transduction by binding to ligands of PD-1 or to PD-1 itself, especially where co-ligation of PD-1 with TCR does not follow such binding, thereby not triggering inhibitory signal transduction through the PD-1 receptor.

Other PD-1 antagonists contemplated by the methods of this invention include antibodies that bind to PD-1 or ligands of PD-1, and other antibodies.

Suitable anti-PD-1 antibodies include, but are not limited to, those described in the following publications: PCT/IL03/00425 (Hardy et al, WO/2003/099196), PCT/JP2006/309606 (Korman et al, WO/2006/121168), PCT/US2008/008925 (Li et al, WO/2009/014708), PCT/JP03/08420 (Honjo et al, WO/2004/004771), PCT/JP04/00549 (Honjo et al, WO/2004/072286), PCT/IB2003/006304 (Collins et al, WO/2004/056875), PCT/US2007/088851 (Ahmed et al, WO/2008/083174), PCT/US2006/026046 (Korman et al, WO/2007/005874), PCT/US2008/084923 (Terrett et al, WO/2009/073533), Berger et al, Clin. Cancer Res., 14:30443051 (2008).

A specific example of an anti-PD-1 antibody is MDX-1106 (see Kosak, US 20070166281 (pub. 19 Jul. 2007) at par. 42), a human anti-PD-1 antibody, preferably administered at a dose of 3 mg/kg.

Exemplary anti-B7-H1 antibodies include, but are not limited to, those described in the following publications: PCT/US06/022423 (WO/2006/133396, pub. 14 Dec. 2006), PCT/US07/088851 (WO/2008/083174, pub. 10 Jul. 2008) US 2006/0110383 (pub. 25 May 2006) A specific example of an anti-B7-H1 antibody is MDX-1105 (WO/2007/005874, published 11 Jan. 2007)), a human anti-B7-Hl antibody.

For anti-B7-DC antibodies see U.S. Pat. Nos. 7,411,051, 7,052,694, 7,390,888, and U.S. Published Application No. 2006/0099203.

The antibody can be a bi-specific antibody that includes an antibody that binds to the PD-1 receptor bridged to an antibody that binds to a ligand of PD-1, such as B7-H1. In some embodiments, the PD-1 binding portion reduces or inhibits signal transduction through the PD-1 receptor.

Other exemplary PD-1 receptor antagonists include, but are not limited to B7-DC polypeptides, including homologs and variants of these, as well as active fragments of any of the foregoing, and fusion proteins that incorporate any of these. In a preferred embodiment, the fusion protein comprises the soluble portion of B7-DC coupled to the Fc portion of an antibody, such as human IgG, and does not incorporate all or part of the transmembrane portion of human B7-DC.

The PD-1 antagonist can also be a fragment of a mammalian B7-H1, preferably from mouse or primate, preferably human, wherein the fragment binds to and blocks PD-1 but does not result in inhibitory signal transduction through PD-1. The fragments can also be part of a fusion protein, for example an Ig fusion protein.

Other useful polypeptides PD-1 antagonists include those that bind to the ligands of the PD-1 receptor. These include the PD-1 receptor protein, or soluble fragments thereof, which can bind to the PD-1 ligands, such as B7-H1 or B7-DC, and prevent binding to the endogenous PD-1 receptor, thereby preventing inhibitory signal transduction. B7-H1 has also been shown to bind the protein B7.1 (Butte et al, Immunity, Vol. 27, pp. 1 11-122, (2007)). Such fragments also include the soluble ECD portion of the PD-1 protein that includes mutations, such as the A99L mutation, that increases binding to the natural ligands (Molnar et al, PNAS, 105: 10483-10488 (2008)). B7-1 or soluble fragments thereof, which can bind to the B7-H1 ligand and prevent binding to the endogenous PD-1 receptor, thereby preventing inhibitory signal transduction, are also useful.

PD-1 and B7-H1 anti-sense nucleic acids, both DNA and RNA, as well as siRNA molecules can also be PD-1 antagonists. Such anti-sense molecules prevent expression of PD-1 on T cells as well as production of T cell ligands, such as B7-H1, PD-L1 and/or PD-L2. For example, siRNA (for example, of about 21 nucleotides in length, which is specific for the gene encoding PD-1, or encoding a PD-1 ligand, and which oligonucleotides can be readily purchased commercially) complexed with carriers, such as polyethyleneimine (see Cubillos-Ruiz et al, J. Clin. Invest. 119(8): 2231-2244 (2009), are readily taken up by cells that express PD-1 as well as ligands of PD-1 and reduce expression of these receptors and ligands to achieve a decrease in inhibitory signal transduction in T cells, thereby activating T cells.

b. CTLA-4 Antagonists

Other molecules useful in mediating the effects of T cells in an immune response are also contemplated as active agents. For example, in some embodiments, the molecule is an agent binds to an immune response mediating molecule that is not PD-1. In a preferred embodiment, the molecule is an antagonist of CTLA4, for example an antagonistic anti-CTLA4 antibody. An example of an anti-CTLA4 antibody contemplated for use in the methods of the invention includes an antibody as described in PCT/US2006/043690 (Fischkoff et al, WO/2007/056539).

Dosages for anti-PD-1, anti-B7-H1, and anti-CTLA4 antibody, are known in the art and can be in the range of 0.1 to 100 mg/kg, with shorter ranges of 1 to 50 mg/kg preferred and ranges of 10 to 20 mg/kg being more preferred. An appropriate dose for a human subject is between 5 and 15 mg/kg, with 10 mg/kg of antibody (for example, human anti-PD-1 antibody, like MDX-1106) most preferred.

Specific examples of an anti-CTLA4 antibody useful in the methods of the invention are Ipilimumab, also known as MDX-010 or MDX-101, a human anti-CTLA4 antibody, preferably administered at a dose of about 10 mg/kg, and Tremelimumab a human anti-CTLA4 antibody, preferably administered at a dose of about 15 mg/kg. See also Sammartino, et al, Clinical Kidney Journal, 3(2): 135-137 (2010), published online December 2009.

In other embodiments, the antagonist is a small molecule. A series of small organic compounds have been shown to bind to the B7-1 ligand to prevent binding to CTLA4 (see Erbe et al, J. Biol. Chem., 277:7363-7368 (2002). Such small organics could be administered alone or together with an anti-CTLA4 antibody to reduce inhibitory signal transduction of T cells.

2. Immunosuppressants

In some embodiments, the additional active agent is one that is known in the art for treatment of inflammation, inflammatory responses, autoimmune diseases and disorders, etc.

Additional therapeutic agents include, but are not limited to, immunosuppressive agents (e.g., antibodies against other lymphocyte surface markers (e.g., CD40, alpha-4 integrin) or against cytokines), other fusion proteins (e.g., CTLA-4-Ig (ORENCIA®), TNFR-Ig (ENBREL®)), TNF-α blockers such as ENBREL, REMICADE, CIMZIA and HUMIRA, cyclophosphamide (CTX) (i.e. ENDOXAN®, CYTOXAN®, NEOSAR®, PROCYTOX®, REVIMMUNE™), methotrexate (MTX) (i.e. RHEUMATREX®, TREXALL®), belimumab (i.e. BENLYSTA®), or other immunosuppressive drugs (e.g., cyclosporin A, FK506-like compounds, rapamycin compounds, or steroids), anti-proliferatives, cytotoxic agents, or other compounds that may assist in immunosuppression.

In some embodiments, the additional therapeutic agent functions to inhibit or reduce T cell activation and cytokine production through a separate pathway. In one such embodiment, the additional therapeutic agent is a CTLA-4 fusion protein, such as CTLA-4 Ig (abatacept). CTLA-4 Ig fusion proteins compete with the co-stimulatory receptor, CD28, on T cells for binding to CD80/CD86 (B7-1/B7-2) on antigen presenting cells, and thus function to inhibit T cell activation. In some embodiments, the additional therapeutic agent is a CTLA-4-Ig fusion protein known as belatacept. Belatacept contains two amino acid substitutions (L104E and A29Y) that markedly increase its avidity to CD86 in vivo. In another embodiment, the additional therapeutic agent is Maxy-4.

In another embodiment, the second therapeutic agent preferentially treats chronic transplant rejection or GvHD, whereby the treatment regimen effectively targets both acute and chronic transplant rejection or GvHD. In another embodiment the second therapeutic is a TNF-α blocker.

In another embodiment, the second therapeutic agent increases the amount of adenosine in the serum, see, for example, WO 08/147482. In some embodiments, the second therapeutic is CD73-Ig, recombinant CD73, or another agent (e.g. a cytokine or monoclonal antibody or small molecule) that increases the expression of CD73, see for example WO 04/084933. In another embodiment the second therapeutic agent is Interferon-beta.

In some embodiments, the compositions are used in combination or succession with compounds that increase Treg activity or production. Exemplary Treg enhancing agents include but are not limited to glucocorticoid fluticasone, salmeteroal, antibodies to IL-12, IFNγ, and IL-4; vitamin D3, and dexamethasone, and combinations thereof. Antibodies to other proinflammatory molecules can also be used in combination or alternation with the disclosed compositions. For example, antibodies can bind to IL-6, IL-23, IL-22 or IL-21.

In some embodiments, the second or more active agent is a rapamycin compound. As used herein the term “rapamycin compound” includes the neutral tricyclic compound rapamycin, rapamycin derivatives, rapamycin analogs, and other macrolide compounds which are thought to have the same mechanism of action as rapamycin (e.g., inhibition of cytokine function). The language “rapamycin compounds” includes compounds with structural similarity to rapamycin, e.g., compounds with a similar macrocyclic structure, which have been modified to enhance their therapeutic effectiveness. Exemplary Rapamycin compounds are known in the art.

In some embodiments, the second or more active agent is an FK506-like compound. The phrase “FK506-like compounds” includes FK506, and FK506 derivatives and analogs, e.g., compounds with structural similarity to FK506, e.g., compounds with a similar macrocyclic structure which have been modified to enhance their therapeutic effectiveness. Examples of FK506-like compounds are known in the art. Preferably, the language “rapamycin compound” as used herein does not include FK506-like compounds.

Other suitable therapeutics include, but are not limited to, anti-inflammatory agents. The anti-inflammatory agent can be non-steroidal, steroidal, or a combination thereof. One embodiment provides oral compositions containing about 1% (w/w) to about 5% (w/w), typically about 2.5% (w/w) or an anti-inflammatory agent. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed.

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

### III. Methods of Manufacture

A. Proteins

Isolated polypeptides including the disclosed proteins, peptides, and fusion proteins can be obtained by, for example, chemical synthesis or by recombinant production in a host cell. To recombinantly produce a polypeptide, a nucleic acid containing a nucleotide sequence encoding the polypeptide can be used to transform, transduce, or transfect a bacterial or eukaryotic host cell (e.g., an insect, yeast, or mammalian cell). In general, nucleic acid constructs include a regulatory sequence operably linked to a nucleotide sequence encoding the polypeptide. Regulatory sequences (also referred to herein as expression control sequences) typically do not encode a gene product, but instead affect the expression of the nucleic acid sequences to which they are operably linked.

In the Examples below, fusion proteins are produced recombinantly in HEK cells. Other suitable reagents and methods of use thereof for producing fusion proteins are known in the art.

Useful prokaryotic and eukaryotic systems for expressing and producing polypeptides are well known in the art include, for example, Escherichia coli strains such as BL-21, and cultured mammalian cells such as CHO cells.

In eukaryotic host cells, a number of viral-based expression systems can be utilized to express polypeptides. Viral based expression systems are well known in the art and include, but are not limited to, baculoviral, SV40, retroviral, or vaccinia based viral vectors.

Mammalian cell lines that stably express variant costimulatory polypeptides can be produced using expression vectors with appropriate control elements and a selectable marker. For example, the eukaryotic expression vectors pCR3.1 (Invitrogen Life Technologies) and p91023(B) (see Wong et al. (1985) Science 228:810-815) are suitable for expression of polypeptides in, for example, Chinese hamster ovary (CHO) cells, COS-1 cells, human embryonic kidney 293 cells, NIH3T3 cells, BHK21 cells, MDCK cells, and human vascular endothelial cells (HUVEC). Additional suitable expression systems include the GS Gene Expression System™ available through Lonza Group Ltd.

Following introduction of an expression vector by electroporation, lipofection, calcium phosphate, or calcium chloride co-precipitation, DEAE dextran, or other suitable transfection method, stable cell lines can be selected (e.g., by antibiotic resistance to G418, kanamycin, or hygromycin). The transfected cells can be cultured such that the polypeptide of interest is expressed, and the polypeptide can be recovered from, for example, the cell culture supernatant or from lysed cells. Alternatively, a polypeptide can be produced by (a) ligating amplified sequences into a mammalian expression vector such as pcDNA3 (Invitrogen Life Technologies), and (b) transcribing and translating in vitro using wheat germ extract or rabbit reticulocyte lysate.

Polypeptides can be isolated using, for example, chromatographic methods such as DEAE ion exchange, gel filtration, and hydroxylapatite chromatography. For example, a polypeptide in a cell culture supernatant or a cytoplasmic extract can be isolated using a protein G column. In some embodiments, polypeptides can be “engineered” to contain an amino acid sequence that allows the polypeptides to be captured onto an affinity matrix. For example, a tag such as c-myc, hemagglutinin, polyhistidine, or Flag™ (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase. Immunoaffinity chromatography also can be used to purify costimulatory polypeptides.

Methods for introducing random mutations to produce variant polypeptides are known in the art. Random peptide display libraries can be used to screen for peptides having the desired activity. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially.

B. Nucleic Acid Molecules

Isolated nucleic acid molecules can be produced by standard techniques, including, without limitation, common molecular cloning and chemical nucleic acid synthesis techniques. For example, polymerase chain reaction (PCR) techniques can be used to obtain an isolated nucleic acid encoding a polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also obtained by mutagenesis. Nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992. Examples of amino acid positions that can be modified include those described herein.

### IV. Methods of Treatment

The disclosed compositions including a chaperone-antigen alone or in combination with an adjuvant, or nucleic acid molecule encoding the foregoing, and/or an additional immunotherapeutic agent can be administered in an effective amount to induce, increase or enhance an immune response. The “immune response” typically refers to responses that induce, increase, induce, or perpetuate the activation or efficiency of innate or adaptive immunity. The compositions can be administered in the absence of other adjuvants may be used to promote tolerance rather than immunity, e.g., to an allergen or autoimmune antigen. The composition can be delivered parenterally (by subcutaneous, intradermal, or intramuscular injection) through the lymphatics, or by systemic administration through the circulatory system. In some embodiments, a chaperone-antigen, an adjuvant, and/or an additional immunotherapeutic agent are administered in the same manner or route. In other embodiments, the different compositions are administered in two or more different manners or routes.

In some embodiments, the compositions are delivered non-systemically. In the most preferred embodiments, at least the chaperone-antigen is delivered locally, for example, by subcutaneous injection. In some embodiments, the composition is administered at a site adjacent to or leading to one or more lymph nodes which are close to the site in need of an immune response (i.e., close to a tumor or site of infection). In some embodiments, the composition is injected into the muscle. For example, in some embodiments, nucleic acids encoding the chaperone-antigen is electroporated into muscle. In some embodiments, the composition is administered in multiple doses at various locations throughout the body. The composition can also be administered directly to a site in need of an immune response (e.g., a tumor or site of infection).

A. Methods of Increasing an Immune Response

The immune response can be induced, increased, or enhanced by the composition compared to a control, for example an immune response in a subject induced, increased, or enhanced by the peptide antigen in the absence of chaperone. Thus, the compositions and methods can be used to induce or increase an immune activating immune response. In some embodiments, the composition reduces inactivation and/or prolong activation of T cells (i.e., increase antigen-specific proliferation of T cells, enhance cytokine production by T cells, stimulate differentiation ad effector functions of T cells and/or promote T cell survival) or overcome T cell exhaustion and/or anergy.

The chaperone-antigen can be used, for example, to induce an immune response, when administering the peptide antigen alone is ineffectual. The chaperone-antigen can also be used to enhance or improve the immune response compared to administering cargo alone. In some embodiments, the chaperone-antigen may reduce the dosage required to induce, increase, or enhance an immune response; or reduce the time needed for the immune system to respond following administration.

Chaperone-antigen may be administered as part of prophylactic vaccines or immunogenic compositions which confer resistance in a subject to subsequent exposure to infectious agents, or as part of therapeutic vaccines, which can be used to initiate or enhance a subject's immune response to a pre-existing antigen, such as a viral antigen in a subject infected with a virus or with cancer.

The desired outcome of a prophylactic or therapeutic immune response may vary according to the disease or condition to be treated, or according to principles well known in the art. For example, an immune response against an infectious agent may completely prevent colonization and replication of an infectious agent, affecting “sterile immunity” and the absence of any disease symptoms. However, a vaccine against infectious agents may be considered effective if it reduces the number, severity or duration of symptoms; if it reduces the number of individuals in a population with symptoms; or reduces the transmission of an infectious agent. Similarly, immune responses against cancer, allergens or infectious agents may completely treat a disease, may alleviate symptoms, or may be one facet in an overall therapeutic intervention against a disease.

The chaperone-antigen can induce an improved effector cell response such as a CD4 or CD8 T-cell immune response, against at least one of the component antigen(s) or antigenic compositions compared to the effector cell response obtained with the corresponding composition without the chaperone. The term “improved effector cell response” refers to a higher effector cell response such as a CD8 or CD4 response obtained in a human patient after administration of a composition having a chaperone-antigen than that obtained after administration of the same composition without a chaperone.

The improved effector cell response can be obtained in an immunologically unprimed patient, i.e. a patient who is seronegative to the antigen. This seronegativity may be the result of the patient having never faced the antigen (so-called “naïve” patient) or, alternatively, having failed to respond to the antigen once encountered. In some embodiments, the improved effector cell response is obtained in an immunocompromised subject.

The improved effector cell response can be assessed by measuring, for example, the number of cells producing any of the following cytokines: (1) cells producing at least two different cytokines (CD40L, IL-2, IFN-gamma, TNF-alpha); (2) cells producing at least CD40L and another cytokine (IL-2, TNF-alpha, IFN-gamma); (3) cells producing at least IL-2 and another cytokine (CD40L, TNF-alpha, IFN-gamma); (4) cells producing at least IFN-gamma and another cytokine (IL-2, TNF-alpha, CD40L); (5) cells producing at least TNF-alpha and another cytokine (IL-2, CD40L, IFN-gamma); and (6) cell producing at least IFN-gamma.

An improved effector cell response is present when cells producing any of the above cytokines will be in a higher amount following administration of the chaperone-antigen compared to control as discussed above.

In a preferred embodiment, the composition increases the number of T cells producing IFN-gamma, TNF-alpha, or a combination thereof, or increases the production of IFN-gamma, TNF-alpha, or a combination thereof in the existing T cells.

In some embodiments, the administration of the immunogenic composition alternatively or additionally induces an improved B-memory cell response in patients administered chaperone-antigen compared to a control. An improved B-memory cell response is intended to mean an increased frequency of peripheral blood B lymphocytes capable of differentiation into antibody-secreting plasma cells upon antigen encounter as measured by stimulation of in vitro differentiation.

In some embodiments, the composition increases the primary immune response as well as the CD8 response. The administration of the composition induces an improved CD4 T-cell, or CD8 T-cell immune response against a specific antigen compared to a control. This method may allow for inducing a CD4 T cell response which is more persistent in time.

Preferably the CD4 T-cell immune response, such as the improved CD4 T-cell immune response obtained in an unprimed subject, involves the induction of a cross-reactive CD4 T helper response. In particular, the amount of cross-reactive CD4 T cells is increased. The term “cross-reactive” CD4 response refers to CD4 T-cell targeting shared epitopes for example between influenza strains.

B. Tolerance

The compositions and methods disclosed herein can also be used to promote tolerance. Tolerogenic therapy aims to induce immune tolerance where there is pathological or undesirable activation of the normal immune response.

Tolerogenic vaccines deliver antigens with the purpose of suppressing immune responses (e.g., induce or increase a suppressive immune response) and promoting robust long-term antigen-specific immune tolerance. For example, Incomplete Freund's Adjuvant (IFA) mixed with antigenic peptides stimulates Treg proliferation (and/or accumulation) and IFA/Insulin peptide prevents type I diabetes onset in susceptible mice, though this approach is ineffective in reversing early onset type I diabetes (Fousteri, G., et al., 53:1958-1970 (2010)). The compositions and methods disclosed herein are also useful for controlling the immune response to an antigen. For example, in some embodiments, the compositions are used as part of a tolerizing vaccine.

A composition typically contains an antigen, or a nucleic acid encoding an antigen as in DNA vaccines, and optionally may include one or more adjuvants. The antigen, for example, a self-antigen, depends on the disease to be treated, and can be determined by one of skill in the art. Exemplary self-antigens and other tolerizing antigens are discussed in more detail above. A chaperone-antigen can be administered in an amount effective to, for example, increase immunosuppression compared to administration of the peptide alone.

C. Diseases to be Treated

1. Cancer

The compositions are useful for stimulating or enhancing an immune response in host for treating cancer. The types of cancer that may be treated with the provided compositions and methods include, but are not limited to, the following: bladder, brain, breast, cervical, colo-rectal, esophageal, kidney, liver, lung, nasopharangeal, pancreatic, prostate, skin, stomach, uterine, ovarian, testicular and hematologic.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The compositions can be administered as an immunogenic composition or as part of vaccine, such as prophylactic vaccines, or therapeutic vaccines, which can be used to initiate or enhance a subject's immune response to a pre-existing antigen, such as a tumor antigen in a subject with cancer.

The desired outcome of a prophylactic or therapeutic immune response may vary according to the disease, according to principles well known in the art. Similarly, immune responses against cancer, may alleviate symptoms, or may be one facet in an overall therapeutic intervention against a disease. For example, administration of the composition may reduce tumor size, or slow tumor growth compared to a control. The stimulation of an immune response against a cancer may be coupled with surgical, chemotherapeutic, radiologic, hormonal and other immunologic approaches in order to affect treatment.

2. Infectious Diseases

The compositions are useful for treating acute or chronic infectious diseases. Because viral infections are cleared primarily by T-cells, an increase in T-cell activity is therapeutically useful in situations where more rapid or thorough clearance of an infective viral agent would be beneficial to an animal or human subject. Thus, the compositions can be administered for the treatment of local or systemic viral infections, including, but not limited to, immunodeficiency (e.g., HIV), papilloma (e.g., HPV), herpes (e.g., HSV), encephalitis, influenza (e.g., human influenza virus A), and common cold (e.g., human rhinovirus) viral infections. For example, pharmaceutical formulations including the composition can be administered topically to treat viral skin diseases such as herpes lesions or shingles, or genital warts. The composition can also be administered to treat systemic viral diseases, including, but not limited to, AIDS, influenza, the common cold, or encephalitis.

Representative infections that can be treated, include but are not limited to infections cause by microorganisms including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Histoplasma, Hyphomicrobium, Legionella, Leishmania, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, Yersinia, Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Plasmodium vivax, Trypanosoma brucei, Entamoeba histolytica, Toxoplasma gondii, Trichomonas vaginalis and Schistosoma mansoni.

In some embodiments, the type of disease to be treated or prevented is a chronic infectious disease caused by a bacterium, virus, protozoan, helminth, or other microbial pathogen that enters intracellularly and is attacked, e.g., by cytotoxic T lymphocytes.

In a preferred embodiment, infections to be treated are chronic infections cause by a hepatitis virus, a human immunodeficiency virus (HIV), a human T-lymphotrophic virus (HTLV), a herpes virus, an Epstein-Barr virus, or a human papilloma virus.

3. Subjects in Need of Tolerance

The compositions that increase tolerance disclosed herein can be used to inhibit immune-mediated tissue destruction for example in a setting of inflammatory responses, autoimmune and allergic diseases, and transplant rejection.

a. Inflammatory and Autoimmune Disorders

In certain embodiments, the disclosed compositions are used to treat an inflammatory response or autoimmune disorder in a subject. For example, the disclosed methods can be used to prophylactically or therapeutically inhibit, reduce, alleviate, or permanently reverse one or more symptoms of an inflammatory response or autoimmune disorder. An inflammatory response or autoimmune disorder can be inhibited or reduced in a subject by administering to the subject an effective amount of a composition in vivo, or cells modulated by the composition ex vivo.

Representative inflammatory responses and autoimmune diseases that can be inhibited or treated include, but are not limited to, rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Bechet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

b. Transplant Rejection

In another embodiment, the disclosed compositions and methods for inducing or perpetuating a suppressive immune response can be used prophylactically or therapeutically to reduce or inhibit graft rejection or graft verse host disease. Transplant rejection occurs when a transplanted organ or tissue is not accepted by the body of the transplant recipient. Typically rejection occurs because the immune system of the recipient attacks the transplanted organ or tissue. The disclosed methods can be used to promote immune tolerance of the transplant or graft by the receipt by administering to the subject an effective amount of a composition in vivo, or cells modulated by the composition ex vivo.

i. Transplants

The transplanted material can be cells, tissues, organs, limbs, digits or a portion of the body, for example the human body. The transplants are typically allogenic or xenogenic. The disclosed compositions are administered to a subject in an effective amount to reduce or inhibit transplant rejection. The compositions can be administered systemically or locally by any acceptable route of administration. In some embodiments, the compositions are administered to a site of transplantation prior to, at the time of, or following transplantation. In one embodiment, compositions are administered to a site of transplantation parenterally, such as by subcutaneous injection.

In other embodiments, the compositions are administered directly to cells, tissue or organ to be transplanted ex vivo. In one embodiment, the transplant material is contacted with the compositions prior to transplantation, after transplantation, or both.

In other embodiments, the compositions are administered to immune tissues or organs, such as lymph nodes or the spleen.

The transplant material can also be treated with enzymes or other materials that remove cell surface proteins, carbohydrates, or lipids that are known or suspected of being involved with immune responses such as transplant rejection.

(a). Cells

Populations of any types of cells can be transplanted into a subject. The cells can be homogenous or heterogenous. Heterogeneous means the cell population contains more than one type of cell. Exemplary cells include progenitor cells such as stem cells and pluripotent cells which can be harvested from a donor and transplanted into a subject. The cells are optionally treated prior to transplantation as mention above.

(b). Tissues

Any tissue can be used as a transplant. Exemplary tissues include skin, adipose tissue, cardiovascular tissue such as veins, arteries, capillaries, valves; neural tissue, bone marrow, pulmonary tissue, ocular tissue such as corneas and lens, cartilage, bone, and mucosal tissue. The tissue can be modified as discussed above.

(c). Organs

Exemplary organs that can be used for transplant include, but are not limited to kidney, liver, heart, spleen, bladder, lung, stomach, eye, tongue, pancreas, intestine, etc. The organ to be transplanted can also be modified prior to transplantation as discussed above.

One embodiment provides a method of inhibiting or reducing chronic transplant rejection in a subject by administering an effective amount of the composition to inhibit or reduce chronic transplant rejection relative to a control.

ii. Graft-Versus-Host Disease (GVHD)

The disclosed compositions and methods can be used to treat graft-versus-host disease (GVHD) by administering an effective amount of the composition to alleviate one or more symptoms associated with GVHD. GVHD is a major complication associated with allogeneic hematopoietic stem cell transplantation in which functional immune cells in the transplanted marrow recognize the recipient as “foreign” and mount an immunologic attack. It can also take place in a blood transfusion under certain circumstances. Symptoms of GVD include skin rash or change in skin color or texture, diarrhea, nausea, abnormal liver function, yellowing of the skin, increased susceptibility to infection, dry, irritated eyes, and sensitive or dry mouth.

In another embodiment, the disclosed compositions and methods for inducing or perpetuating a suppressive immune response can be used prophylactically or therapeutically to suppress allergies and/or asthma and/or inflammation. Allergies and/or asthma and/or inflammation can be suppressed, inhibited or reduced in a subject by administering to the subject an effective amount of a composition that promotes an immune suppressive immune response or tolerance as described above.

C. Treatment Regimens

The Examples below shows that the chaperone-antigen and adjuvant can be administered as a vaccine that includes a first (“prime”) and second (“boost”) administration. Thus in some embodiments, a vaccine is administered 2, 3, 4, or more times, for example, 1, 2, 3, 4, 5, 6, 7, 8, 9, 10, 11, 12, 13, 14, or 15 days, weeks, months, or years apart.

In some embodiments, the methods include administration of another therapeutic agent, for example an immunotherapeutic agent. In the Example below, the immunotherapeutic agent was administered once a week for 5 weeks. Dosage regimens or cycles of the agents can be completely or partially overlapping, or can be sequential.

### EXAMPLES

### Example 1: Protein-Chaperoned Vaccines Traffic to the Lymph Nodes and Enhance T Cell Vaccines

Materials and Methods

Mouse Model

The experimental mouse model includes C57BL/6 mice subcutaneously injected with antigen and adjuvant. During vaccination, antigen and adjuvant are co-injected on separate molecules. Vaccines are typically performed in a prime/boost schedule: mice are primed on day 0 with antigen and adjuvant, boosted on day 14 homologously, and bled on day 20, when either tetramer staining or intracellular cytokine staining are employed to measure vaccine response. One experiment utilized the alternative schedule of prime at day 0, boost at day 13, and bled at day 19. The results indicate that the alternative schedule does not substantially alter the results.

In tetramer analysis, vaccine response was measured by collecting 100 ul of peripheral blood from vaccinated mice 6 days following boost, lysing red blood cells with ACK lysing buffer, and using an E7 tetramer to measure the fraction of circulating CD8 T cells that are antigen-specific.

For intracellular cytokine analysis, following prime and boost, 100 ul of peripheral blood was collected from vaccinated and naïve mice. Red blood cells were lysed using ACK lysing buffer. Remaining cells were restimulated ex vivo with native peptide ligand for 24 hours. For the final 6 hours of restimulation, brefeldin A was added to culture media. Cells were then intracellularly stained for TNFa and IFNg to determine the frequency of stimulated T cells among all CD8 T cells.

For analysis of lymph node targeting at −8 hours mice are injected with 100 ug MSA, 85 ug Fc, or 3 ug peptide labeled with FAM (no adjuvant). At 0 hours inguinal LN are dissected out and measure with IVIS® (in vivo imaging system) and flow cytometry. More specifically, with respect to the experiment with results of which are shown in FIG. 4A, MSA-E7 long fusions were labeled on their free surface-exposed lysines with FAM-NHS, and all proteins were confirmed to have equivalent degrees of labeling relative to each other and free FAM-E7 long peptide. 6 ug peptide equivalent was subcutaneously injected in the tail base of C57BL/6 mice. 8 hours later, draining inguinal lymph nodes were extracted and imaged on an IVIS system (excitation 500 nm, emission 540 nm). With respect to the experiment with results of which are shown in FIG. 4B MSA- and Fc-antigen fusions were labeled on their free surface-exposed lysines with FAM-NHS, and all proteins were confirmed to have equivalent degrees of labeling relative to each other and free FAM-E7 long peptide. 3 ug peptide equivalent were subcutaneously injected in the tail base of C57BL/6 mice. 8 hours later, draining inguinal lymph nodes were extracted and imaged on an IVIS system (excitation 500 nm, emission 540 nm).

Antigens

The model antigens include HPV E7₃₈₋₅₇ (IDGPAGQAEPDRAHYNIVTF (SEQ ID NO:1)), human carcinoembryonic antigen (CEA)₅₆₇₋₅₈₄

(SEQ ID NO: 2)

(RAYVSGIQNSVSANRSDP),

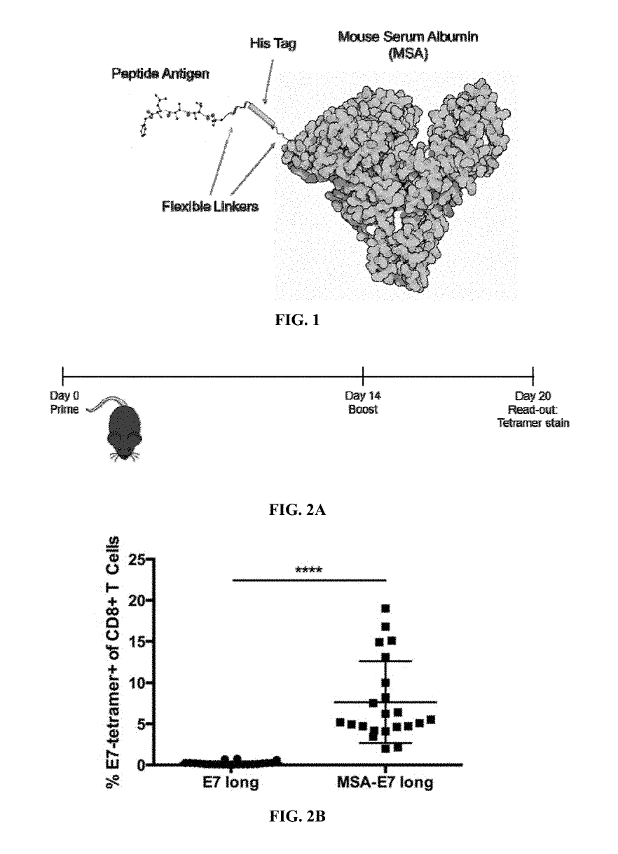

and altered peptide ligand forms of mouse tyrosinase-related protein 1 (Trp1)₄₅₅₋₄₆₃ (TAPDNLGYM (SEQ ID NO:3) and TAPDNLGYA (SEQ ID NO:4)). All peptides are fused to the C termini of protein carriers via a GGGS (SEQ ID NO: 13) linker followed by a His tag for purification, another GGGS (SEQ ID NO: 13) linker, and a peptide antigen (see, e.g., FIG. 1). EGP is also the name of an altered peptide sequence of a melanocyte-associated antigen named gp100 (native sequence: AVGALEGSRNQDWLGVPRQL (SEQ ID NO:20), altered sequence: AVGALEGPRNQDWLGVPRQL (SEQ ID NO:21)).

MSA-Trp1, MSA-EGP, and MSA-CEA antigen fusions were generated in the same fashion as MSA-E7 long. In the case of Trp1, an altered peptide ligand form of the Trp1 antigen (where the C-terminal anchor residue A is replaced with M) was utilized (native sequence: TAPDNLGYM (SEQ ID NO:3), altered sequence: TAPDNLGYA (SEQ ID NO:4)).

In the case of CEA, the native antigen sequence was used:

(SEQ ID NO: 2)

RAYVSGIQNSVSANRSDP.

Protein Carriers

The vaccines utilized seven different protein carriers: transthyretin (TTR), mouse serum albumin (MSA), the wild-type Fc portion of mouse IgG2a (FcWT), a mutant form of the Fc portion of mouse IgG2a (G236R and L328R) that abrogates interaction with Fcγ receptors (FcKO), and three variants of an archae-derived DNA-binding protein named sso7d. Via mutagenesis, a charge-reduced sso7d variant named rcSso7d was developed. A yeast surface display library was generated using rcSso7d as a scaffold, and two MSA binders were isolated, named M11.1.2 and M18.2.5.

TTR

(SEQ ID NO: 26)

GPAGAGESKCPLMVKVLDAVRGSPAVDVAVKVFKKTSEGSWEPFASGKTA
ESGELHGLTTDEKFVEGVYRVELDTKSYWKTLGISPFHEFADVVFTANDS
GHRHYTIAALLSPYSYSTTAVVSNPQN
MSA

(SEQ ID NO: 6)

EAHKSEIAHRYNDLGEQHFKGLVLIAFSQYLQKCSYDEHAKLVQEVTDFA
KTCVADESAANCDKSLHTLFGDKLCAIPNLRENYGELADCCTKQEPERNE
CFLQHKDDNPSLPPFERPEAEAMCTSFKENPTTFMGHYLHEVARRHPYFY
APELLYYAEQYNEILTQCCAEADKESCLTPKLDGVKEKALVSSVRQRMKC
SSMQKFGERAFKAWAVARLSQTFPNADFAEITKLATDLTKVNKECCHGDL
LECADDRAELAKYMCENQATISSKLQTCCDKPLLKKAHCLSEVEHDTMPA
DLPAIAADFVEDQEVCKNYAEAKDVFLGTFLYEYSRRHPDYSVSLLLRLA
KKYEATLEKCCAEANPPACYGTVLAEFQPLVEEPKNLVKTNCDLYEKLGE
YGFQNAILVRYTQKAPQVSTPTLVEAARNLGRVGTKCCTLPEDQRLPCVE
DYLSAILNRVCLLHEKTPVSEHVTKCCSGSLVERRPCFSALTVDETYVPK
EFKAETFTFHSDICTLPEKEKQIKKQTALAELVKHKPKATAEQLKTVMDD
FAQFLDTCCKAADKDTCFSTEGPNLVTRCKDALA (NCBI: P07724
(precursor to MSA))
FcWT

(SEQ ID NO: 7)

EPRVPITQNPCPPLKECPPCAAPDLLGGPSVFIFPPKIKDVLMISLSPMV
TCVVVDVSEDDPDVQISWFVNNVEVHTAQTQTHREDYNSTLRVVSALPIQ
HQDWMSGKEFKCKVNNRALPSPIEKTISKPRGPVRAPQVYVLPPPAEEMT
KKEFSLTCMITGFLPAEIAVDWTSNGRTEQNYKNTATVLDSDGSYFMYSK
LRVQKSTWERGSLFACSVVHEGLHNHLTTKTISRSLGK
FcKO

(SEQ ID NO: 8)

EPRVPITQNPCPPLKECPPCAAPDLLRGPSVFIFPPKIKDVLMISLSPMV
TCVVVDVSEDDPDVQISWFVNNVEVHTAQTQTHREDYNSTLRVVSALPIQ
HQDWMSGKEFKCKVNNRARPSPIEKTISKPRGPVRAPQVYVLPPPAEEMT
KKEFSLTCMITGFLPAEIAVDWTSNGRTEQNYKNTATVLDSDGSYFMYSK
LRVQKSTWERGSLFACSVVHEGLHNHLTTKTISRSLGK
M11.1.2

(SEQ ID NO: 9)

ATVKYTYRGEEKRVDISKIKWVNRWGQHLAFKYDKGGGAAGYGWVSEKDA
PKELLQMLEKR
M18.2.5

(SEQ ID NO: 10)

ATVKFTYRGEEKQVDISKIKWVVRLGQVIMFKYDEGGGASGYGRVSEKDA
PKELLQMLEK
rcSso7d

(SEQ ID NO: 11)

ATVKFTYQGEEKQVDISKIKKVWRVGQMISFTYDEGGGATGRGAVSEKDA
PKELLQMLEKQ

Fusion Protein Preparation

A gene encoding MSA-long protein is inserted into the GWiz plasmid (Genlantis) and transiently transfected into HEK FreeStyle cells to induce protein production. Pure protein product is collected from supernatant using TALON resin (Clontech) to isolate His-tagged proteins. MSA-Trp1 and MSA-CEA antigen fusions were generated in the same fashion as MSA-E7 long. In the case of Trp1, an altered peptide ligand form of the Trp1 antigen (where the C-terminal anchor residue A is replaced with M) was utilized.

Exemplary Fusion Proteins

Mouse TTR-E7 Long

Leader sequence:

(SEQ ID NO: 25)

MRVPAQLLGLLLLWLPGARCA
Mouse TTR:

(SEQ ID NO: 26)

GPAGAGESKCPLMVKVLDAVRGSPAVDVAVKVFKKTSEGSWEPFASGKTA
ESGELHGLTTDEKFVEGVYRVELDTKSYWKTLGISPFHEFADVVFTANDS
GHRHYTIAALLSPYSYSTTAVVSNPQN
Linker:

(SEQ ID NO: 27)

GGGSHHHHHHGGGS
E7 long:

(SEQ ID NO: 1)

IDGPAGQAEPDRAHYNIVTF

Mouse MSA-E7 Long

Leader sequence:

(SEQ ID NO: 25)

MRVPAQLLGLLLLWLPGARCA
MSA:

(SEQ ID NO: 6)

EAHKSEIAHRYNDLGEQHFKGLVLIAFSQYLQKCSYDEHAKLVQEVTDFA
KTCVADESAANCDKSLHTLFGDKLCAIPNLRENYGELADCCTKQEPERNE
CFLQHKDDNPSLPPFERPEAEAMCTSFKENPTTFMGHYLHEVARRHPYFY
APELLYYAEQYNEILTQCCAEADKESCLTPKLDGVKEKALVSSVRQRMKC
SSMQKFGERAFKAWAVARLSQTFPNADFAEITKLATDLTKVNKECCHGDL
LECADDRAELAKYMCENQATISSKLQTCCDKPLLKKAHCLSEVEHDTMPA
DLPAIAADFVEDQEVCKNYAEAKDVFLGTFLYEYSRRHPDYSVSLLLRLA
KKYEATLEKCCAEANPPACYGTVLAEFQPLVEEPKNLVKTNCDLYEKLGE
YGFQNAILVRYTQKAPQVSTPTLVEAARNLGRVGTKCCTLPEDQRLPCVE
DYLSAILNRVCLLHEKTPVSEHVTKCCSGSLVERRPCFSALTVDETYVPK
EFKAETFTFHSDICTLPEKEKQIKKQTALAELVKHKPKATAEQLKTVMDD
FAQFLDTCCKAADKDTCFSTEGPNLVTRCKDALA
Linker:

(SEQ ID NO: 27)

GGGSHHHHHHGGGS
E7 long:

(SEQ ID NO: 1)

IDGPAGQAEPDRAHYNIVTF

Adjuvants

Adjuvants include a lipidated CpG molecule previously described in (Liu 2014) (lipo-CpG), unformulated CpG, polyinosinic:polycytidylic acid (polyIC), and cyclic dinucleotides (CDN). Dosing consists of 3-10 μg of peptide equivalence, 1.2 nmol CpG or lipo-CpG, 50 μg polyIC, and 25-50 μg CDN.

Results

Following protein production of MSA-E7 on TALON resin, protein product was run through a Superdex 200 Increase 10/300 (GE Life Sciences) size exclusion column. MSA-E7 eluted in one peak. Yield in HEK culture: ˜30 mg/ml.

Fusion proteins can improve the potency of otherwise weak peptide antigens in a generalizable fashion.

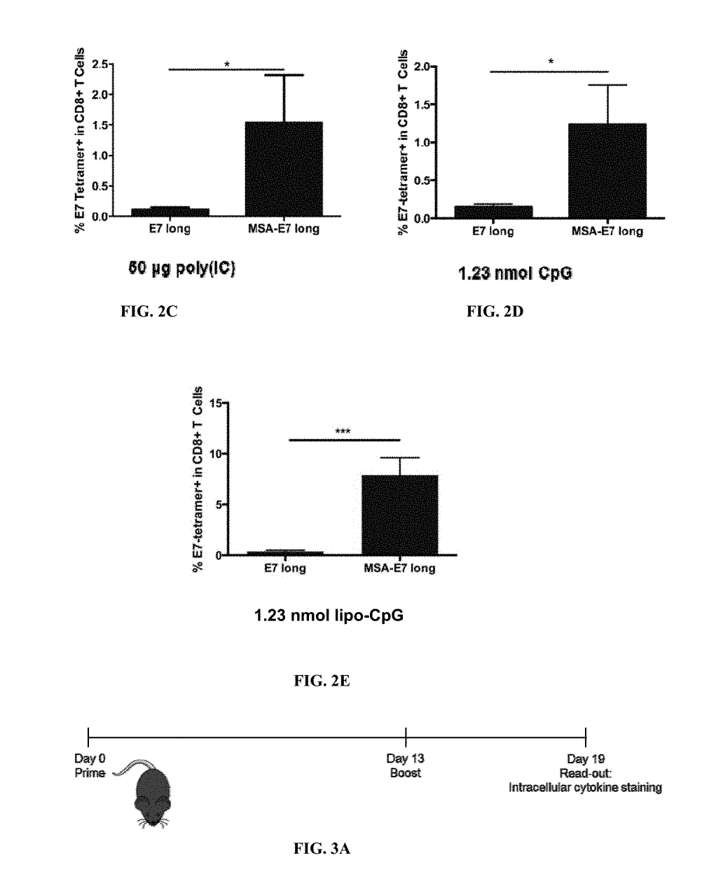

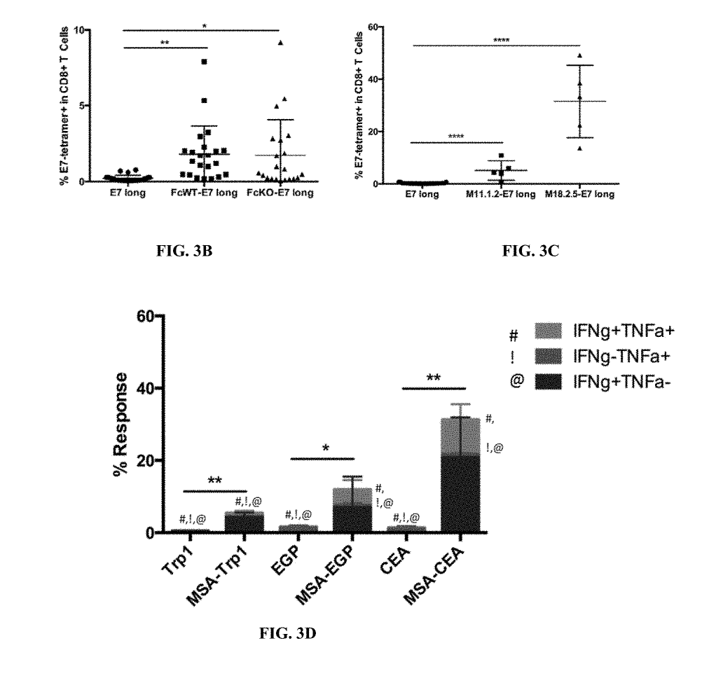

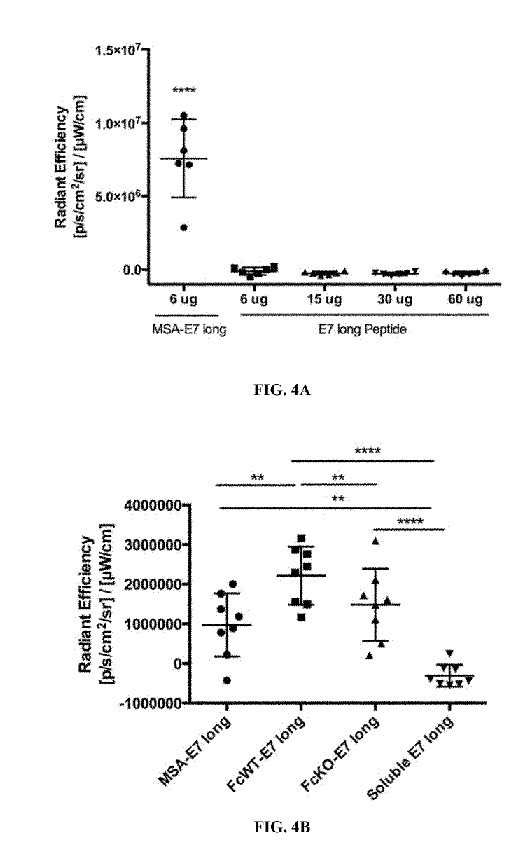

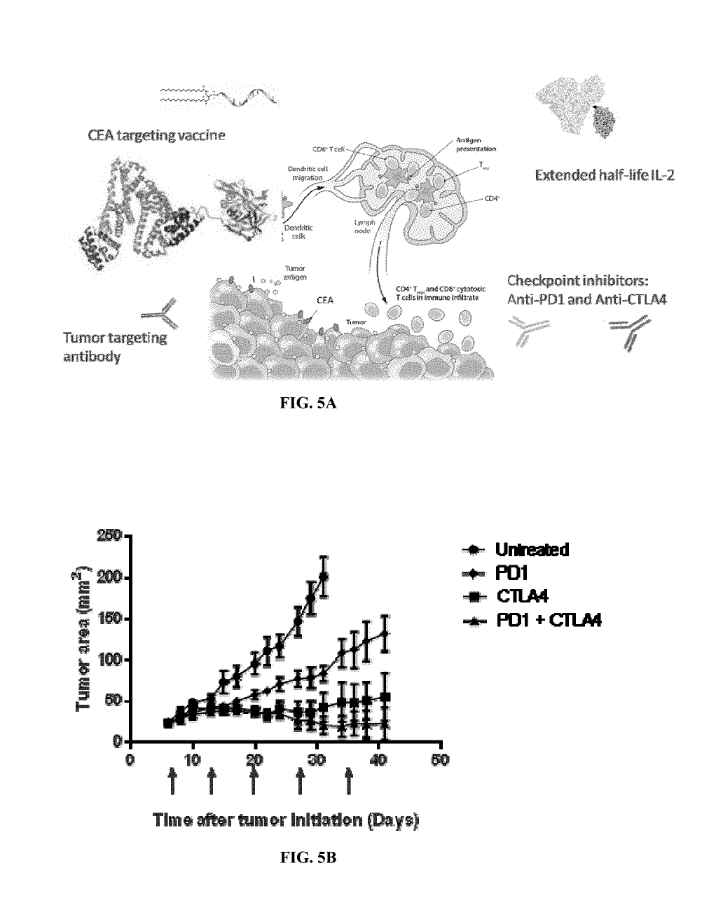

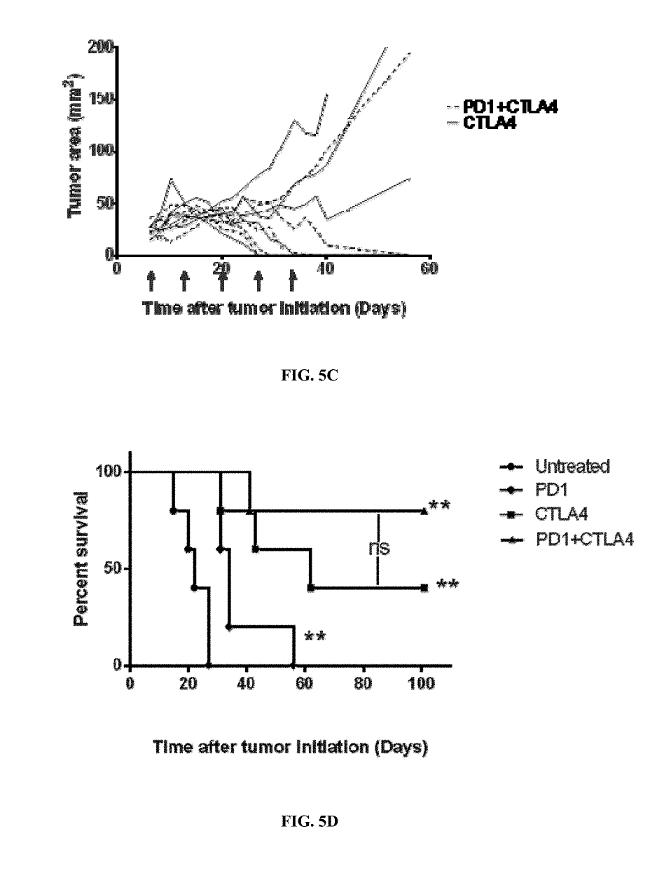

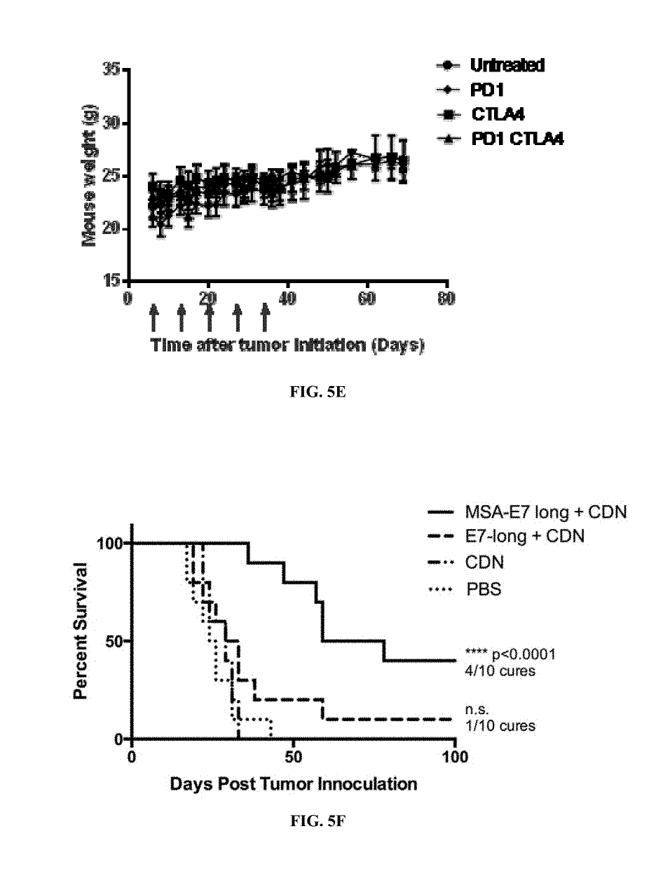

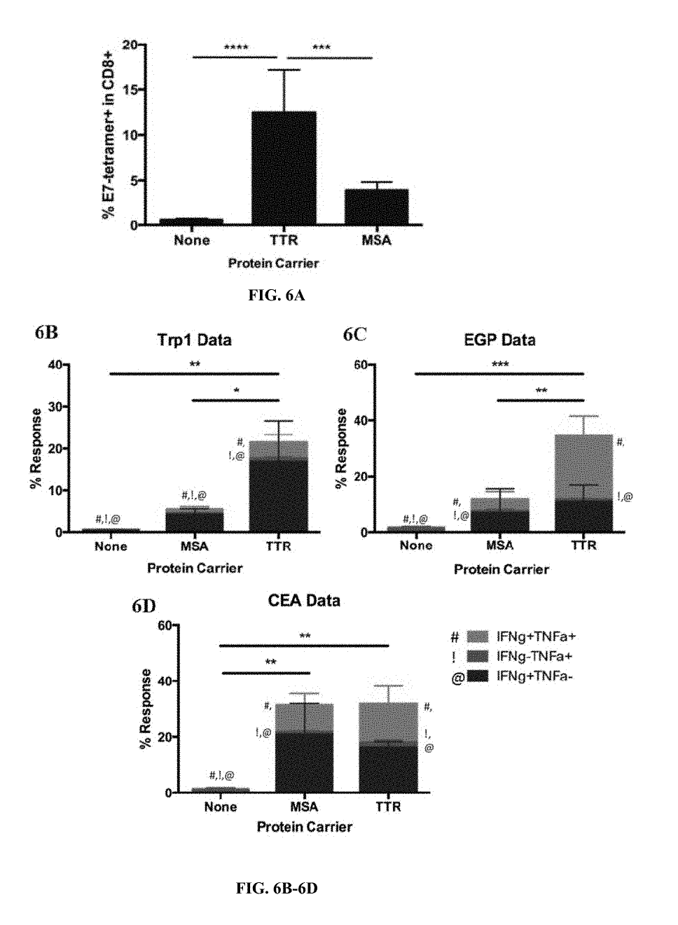

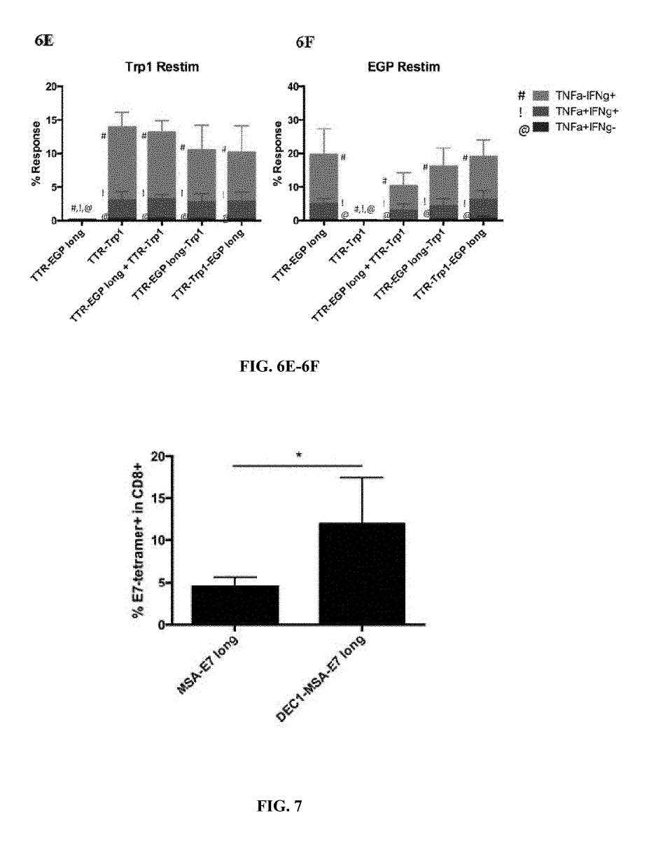

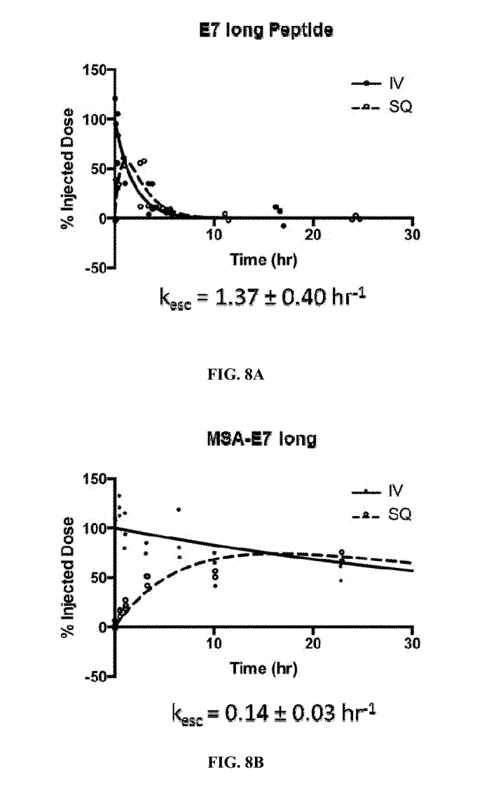

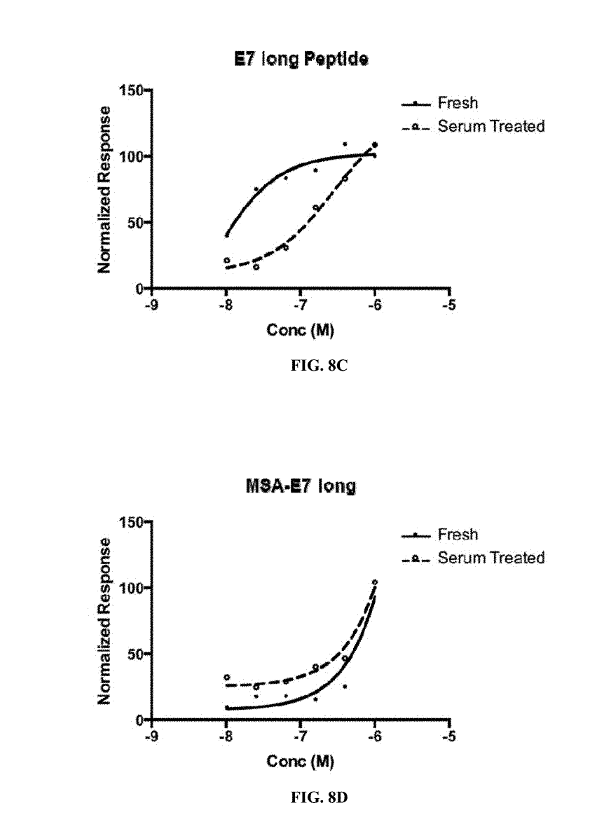

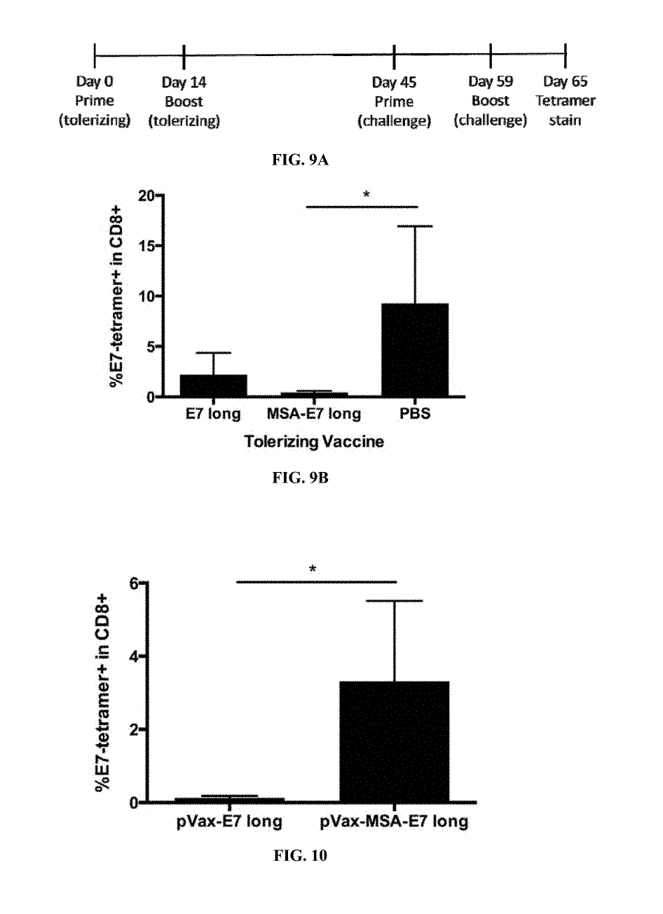

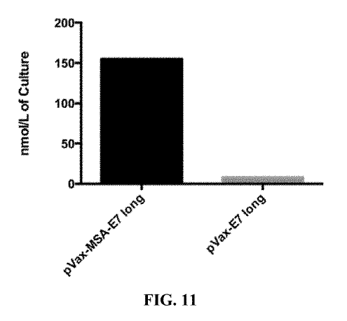

The E7₃₈₋₅₇ peptide and CDN vaccine consistently generates weak responses: following vaccination, less than 0.5% of circulating CD8 T cells are E7-specific (FIG. 2B). MSA fusion dramatically improved the potency of the antigen. MSA-E7₃₈₋₅₇ results in potent responses of approximately 7.5%, closer to the frequency one would expect in a natural infection (FIG. 2B). FcWT, FcKO, M11.1.2, and M18.2.5 all generate T cell responses significantly greater than non-chaperoned peptide (FIG. 3B-3C).

Additionally, the improvement in E7₃₈₋₅₇ has been observed with adjuvants other than CDN, including lipo-CpG, CpG, and polyIC (FIGS. 2C-2E).

This improvement in vaccine potency has also been observed with Trp₁₄₅₅₋₄₆₃, EGP, and CEA₅₆₇₋₅₈₄ (FIG. 3D). In all cases, 25 ug cyclic di-GMP was used as an adjuvant.

MSA- and Fc-antigen fusions were labeled on their free surface-exposed lysines with FAM-NHS, and all proteins were confirmed to have equivalent degrees of labeling relative to each other and free FAM-E7 long peptide. Results are illustrated in FIGS. 4A and 4B. These data show that even 10-fold higher doses of peptide cannot gain access to the LN, whereas MSA fusions easily can do so, even at relatively low doses (FIG. 4A). In no case are Fc-fusions taken up less readily than MSA. This indicates that both MSA and Fc fusions can improve LN uptake (FIG. 4B) relative to control and E7 long peptide alone.

### Example 2: Protein Chaperones Enhance Tumor Regression

Materials and Methods

MSA-CEA Study

Lung adenocarcinoma cell lines were transfected with a human CEA transgene. 1×10⁶ CEA-expressing lung adenocarcinoma cells were implanted subcutaneously into the flanks of transgenic C57BL/6 mice expressing human CEA. On day 6 after tumor initiation, mice were vaccinated subcutaneously at the left and right tail base with 360 μg (equivalent to 10 μg of the CEA 567-584 peptide) of MSA-CEA₅₆₇₋₅₈₄ peptide fusion and 1.2 nmol of lipo-CpG. Immediately after vaccination, mice were treated intra-peritoneally with 30 μg of MSA-IL-2 and 8 mg/kg each of anti-CEA antibody (SM3e, mouse IgG2a isotype), anti-PD1 (Clone RMP1-14, rat IgG2a isotype) and anti-CTLA4 (Clone 9D9, mouse IgG2a isotype). Mice received this combination immunotherapy every 7 days for 5 weeks. Tumor area was measured every other day and mice were sacrificed when tumor area exceeded 200 mm².

MSA-E7 Long Peptide Study

B6 mice were inoculated with 300,000 TC-1 tumor cells derived from mice preimmunized against HPV16 E7 oncoprotein. On days 5, 12, and 17, mice were treated with the described vaccination. Dosing was 50 μg cyclic dinucleotides and 10 μg E7 long peptide or peptide equivalent. Mice were euthanized when their tumors grew to over 100 mm² in area.

Results

MSA-CEA Study

To study the therapeutic effect of fusion peptide based vaccines in combination with other immunotherapy modalities, a murine model of lung adenocarcinoma was utilized. 10⁶ 2677-CEA tumor cells were implanted subcutaneously on Day 0. On day 6 after tumor initiation, 5 weekly treatments of combination immunotherapy were administered (FIG. 5A). The results are presented in FIGS. 5B-5E: Tumor growth curves of cohorts (n=5) treated with AIPV, AICV, AIPCV or untreated (FIG. 5B); individual tumor growth curves of mice that have regressed tumors (FIG. 5C); cumulative survival of tumor bearing mice, death events correspond to tumors of area >150 mm² (FIG. 5D); Mouse body weight tracked over time (n=5) (FIG. 5E).

The acronyms AIPV, AICV, and AIPCV state which elements of the combination therapy are utilized during treatment: “A”—antibody; “I”-extended half-life IL2; “P”—anti-PD1 antibody; “C”—anti-CTLA4 antibody; “V”—vaccine.

MSA-E7 Long Peptide Study

In another experiment, B6 mice were inoculated with 300,000 TC-1 tumor cells. On days 5, 12, and 17, mice were treated with the described vaccination. In the peptide vaccine setting, a subset of outlier mice had delayed tumor outgrowth and 1/10 mice survived, while in the MSA-peptide vaccine setting, all mice delayed tumor outgrowth relative to PBS control and 4/10 mice were cured (FIG. 5F).

### Example 3: Transthyretin (TTR) is Effective as a Protein Chaperon

Materials and Methods

TTR-E7 long was prepared in the same fashion as MSA-E7 long. Because TTR is a tetramer, there are four copies of E7 long peptide cargo per protein. As a result, 25 μg of TTR-E7 long were compared against 100 μg MSA-E7 long in a prime boost vaccination model. Six days after boost, 100 μl of peripheral blood was collected from vaccinated and naïve mice. Red blood cells were lysed using ACK lysing buffer. Remaining cells were labeled with an E7-specific MHC tetramer to measure the fraction of circulating E7-specific CD8 T cells.

TTR-Trp1, TTR-EGP long, and TTR-CEA long were prepared in the same fashion as the MSA-antigen fusions described above. Like in the E7 long experiment, mice were dosed with 1/4 as much TTR-antigen fusion compared to MSA-antigen fusion. In the case of TTR-Trp1 and TTR-EGP long, however, responses were more than doubled when TTR was used as a vaccine carrier. TTR-CEA long was equivalently immunogenic to MSA-CEA long. All vaccines were prime/boost models with 25 μg cyclic dinucleotides used as an adjuvant.

TTR-EGP long-Trp1 was prepared by cloning the genes for TTR followed by EGP long followed by Trp1 in frame on a mammalian expression vector. Similarly, TTR-Trp1-EGP long was cloned but with TTR followed by Trp1 followed by EGP long. Antigens and dosing regimens were as described above.

Results

The Examples above show that Fc and sso7d fusions improve the potency of antigens such as E7. Further experiments show that another protein, transthyretin (TTR), also has the effect of improving vaccine responses, with several different antigens including E7 and the three antigens shown above (FIGS. 6A-6D). In all cases, using TTR as a protein carrier is either equivalently effective to or more effective than using MSA as a protein carrier.

Because TTR is a tetramer, its use as a vaccine carrier opens up the possibility of co-delivering multiple antigens at once. As a proof of concept, TTR was produced in such a way that it co-delivers Trp1 and EGP long on the same protein, resulting in potent immune responses against both antigens independent of antigen orientation. In both cases (TTR-EGP long-Trp1 and TTR-Trp1-EGP) Trp1 responses and EGP responses were similar to equivalent dosing of the corresponding TTR-single antigen fusion and to TTR-EGP long and TTR-Trp1 injected on separate molecules (TTR-EGP long+TTR-Trp1) (FIGS. 6E-6F).

### Example 4: Targeting Dendritic Cells Enhance T Cell Activation

Materials and Methods

Mice were primed on day 0, boosted on day 14, and peripheral blood tetramer stained on day 20. Red blood cells were lysed using ACK lysing buffer. Remaining cells were labeled with an E7-specific MHC tetramer to measure the fraction of circulating E7-specific CD8 T cells.

Results

Another technological improvement comes in the form of targeting of vaccines to antigen-presenting cells. Cross-presenting dendritic cells are known to express an internalizing receptor named DEC-205. Targeting antigen to DEC-205 has been shown to facilitate effective cross presentation and T cell activation. A DEC-205 binding fibronectin called DEC1 was attached to the N-terminal end of MSA-E7 long and found to enhance vaccine potency in vivo (FIG. 7).

### Example 5: Protein Chaperone Fusion Proteins are Preserved in the Subcutaneous Space for a Longer Period of Time than Free Antigen

Materials and Methods

Circulation Study

E7 long peptide and MSA-E7 long protein were labeled with fluorescein and injected into B6 mice either intravenously (IV) or subcutaneously (SQ). Their blood was subsequently monitored for fluorescein signal and compared against a standard curve to measure antigen concentration for 24 hours.

Serum Study

Mice were vaccinated with an irrelevant E7-stimulating vaccine. Splenocytes from vaccinated mice were isolated and pooled and restimulated with a dilution series of E7 long peptide or MSA-E7 long, either fresh from the fridge or following overnight treatment with 10% mouse serum at 37 C. The potency of the immunogen was measured via intracellular cytokine staining to detect IFNγ production.

Results

The mechanisms as to why protein-delivered vaccines outperform naked peptide vaccines were also investigated. The kinetics of antigen in blood following subcutaneous injection are based on two rate constants: the rate of clearance from blood and the rate of escape from the site of injection into the blood. The IV curve was used to measure the clearance rate and the subcutaneous curve to deduce the rate of escape (k_{esc}). The rate of escape of MSA-E7 long fusion is approximately ten-fold slower than the rate of escape of E7 long peptide (FIGS. 8A-8B).

The results indicate that subcutaneous injection of E7 long results in rapid systemic uptake in blood, whereas MSA-E7 long is preserved in the subcutaneous space for a longer period of time, allowing for better lymph node drainage.

Splenocytes from vaccinated mice were also isolated and pooled and restimulated with a dilution series of E7 long peptide or MSA-E7 long, either fresh from the fridge or following overnight treatment with 10% mouse serum at 37 C. Although the potency of E7 long peptide was harmed by serum treatment, the potency of MSA-E7 long was unaffected (FIG. 8C-8D). The results indicate that MSA fusion tends to protect peptide cargo from loss of potency in serum.

### Example 6: Chaperone Protein Improves Tolerogenic Vaccines

Materials and Methods

### Groups:

(1) E7 and no adjuvant intravenously for tolerization; TTR-E7+CDN subcutaneously for challenge.

(2) MSA-E7 and no adjuvant IV for tolerization; TTR-E7+CDN SQ for challenge.

(3) PBS intravenous (IV) for tolerization; TTR-E7+CDN subcutaneous (SQ) for challenge.

Results

A carrier protein strategy can be used in tolerogenic vaccines. A tolerizing assay according to the materials and methods above and the assay design of FIG. 9A was carried out. The results are illustrated in FIG. 9B. In a tolerizing vaccine format, MSA-E7 long fusion protein induced a lower % E7-tetramer+ in CD8+ T cells relative to free E7 long peptide.

### Example 7: Chaperone Protein Strategy can be Used in a DNA Vaccine Setting

Materials and Methods

The sequence for E7 long or MSA-E7 long along with leader sequence and Kozak sequence was cloned into commercially available pVax1 plasmid.

Mice were vaccinated with 25 μg pVax1-E7 long or 40 μg pVax1-MSA-E7 long (equivalent doses by copies of plasmid DNA)

Vaccinations were performed intramuscularly followed by in vivo electroporation.

Mice were primed on day 0, boosted on day 14, tetramer stain read-out on day 21.

Results

A carrier protein strategy enables DNA vaccine immunogenicity. The results are illustrated in FIG. 10.

### Example 8: Chaperone Protein Enhances Antigen Expression

Materials and Methods

pVax1 encoding either E7 long or MSA-E7 long were used to transfect HEK cells in culture fused to a His tag.

A competitive His ELISA was used to quantify levels of expression of either E7 long or MSA-E7 long in the HEK cell supernatant.

Results

Carrier protein strategy improves antigen expression. A show in FIG. 11, using MSA as a carrier protein dramatically improved protein expression. The results may explain why protein chaperone fusion proteins are so effective in a DNA vaccine setting (see Example 7).

The Examples show that in a subcutaneous injection setting, protein chaperones promote efficient lymph node uptake and preserve the bioavailability of antigen to facilitate more efficient T cell priming. The use of protein chaperones also dramatically increases lymph node uptake, improving vaccine responses, and promoting tumor rejection in pre-clinical mouse models.

When used with an adjuvant, peptide chaperone fusion proteins improve cancer survival. When peptide antigens fused to protein chaperones are delivered with an adjuvant (as a vaccine) as part of a combination immunotherapy regimen including (i) a tumor targeting antibody (ii) an extended serum half-life IL-2 (MSA-IL2 previously described in Zhu, et al., Cancer Cell, 27:489-501 (2015)) and (iii) checkpoint inhibitor antibodies like anti-PD-1 and anti-CTLA4, they can elicit potent anti-tumor CD8 T-cell responses and result in the regression of 80% of mice bearing large established tumors.

One receptor-independent explanation for the potency enhancement is improved vaccine trafficking from the injection site to the dLN. Larger macromolecules drain more efficiently, so attaching peptides (˜1 kDa) to MSA (˜70 kDa) may impart an advantage. Indeed, while even high doses of fluorescein-labeled E7 (60 μg) cannot be detected in the dLN via IVIS imaging 8 hours post-injection, labeled MSA-E7 is readily detected even at low doses (3 μg peptide equivalence), and labeled Fc-E7 fusions (˜60 kDa) drain to the LN similarly to MSA-E7. Additionally, fusion tends to protect peptide cargo from loss of potency in serum.

Protein chaperones have advantages that may make them useful in a therapeutic vaccination setting. For example, peptides of certain amino acid sequences may be poorly behaved in solution following solid phase peptide synthesis, interfering with the execution of chemical modifications often performed on peptides to improve their antigenicity and hindering clinical usage. Highly soluble protein chaperones that stabilize peptides improve the ease of manipulation in solution. Additionally, solid phase peptide synthesis has a functional maximum peptide length of roughly 50 amino acids due to limits in coupling efficiency. Recombinantly produced protein-peptide fusions may help overcome this limit and allow for long antigens and/or several antigens to be linked together.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of increasing an immune response or promoting tolerance in a subject in need thereof comprising administering the subject a chaperone-antigen comprising a peptide antigen conjugated or fused to a chaperone protein, or a nucleic acid encoding the chaperone-antigen, in an effective amount to increase lymph node uptake; improve an immune response or promote tolerance; or a combination relative to administering the peptide antigen alone.

2. The method of claim 1, wherein the chaperone-antigen or a nucleic acid encoding the chaperone-antigen is administered non-systemically to the subject.

3. The method of the claim 2, wherein the chaperone-antigen or a nucleic acid encoding the chaperone-antigen is administered locally to the subject.

4. The method of claim 3, wherein the chaperone-antigen or a nucleic acid encoding the chaperone-antigen is administered subcutaneously or intramuscularly.

5. The method of claim 1, wherein the peptide antigen is fused to the chaperone protein to form a fusion protein.

6. The method of claim 5, wherein the fusion protein comprises a linking domain linking the peptide antigen and chaperone protein.

7. The method of claim 6, wherein the linking domain comprises a first flexible linker linked to a purification tag linked to a second flexible linker.

8. The method of claim 1, wherein the chaperone reduces or prevents a loss of potency of the peptide antigen in the presence of the serum.

9. The method of claim 1, wherein the chaperone is sufficiently large to facilitate effective lymph node uptake or is a binder to an endogenous molecule sufficiently large molecular weight to facilitate effective lymph node uptake.

10. The method of claim 1, wherein the chaperone induces little or no immune response in the subject.

11. The method of claim 1, wherein the chaperone is a protein that is endogenous to the subject, or a functional fragment or variant thereof.

12. The method of claim 1, wherein the chaperone is a serum protein.

13. The method of claim 12, wherein the serum protein is selected from the group consisting of albumins, globulins, fibrinogen, regulatory proteins, and clotting factors.

14. The method of claim 1, wherein the chaperone is a transthyretin (TTR), serum albumin, Fc, or a functional fragment or variant thereof.

15. The method of claim 1, wherein the peptide antigen is derived from a virus, bacterium, parasite, plant, protozoan, fungus, tissue or transformed cell.

16. The method of claim 1, wherein the peptide antigen is a neocancer antigen.

17. The method of claim 1, further comprising administering the subject an adjuvant.

18. The method of claim 1, further comprising administering the subject an additional immunotherapeutic agent selected from the group consisting of (i) a tumor targeting antibody, (ii) an extended serum half-life IL-2, (iii) an immune checkpoint inhibitor, or (iv) a combination thereof.

19. A pharmaceutical composition comprising an effective amount of a chaperone-antigen comprising a peptide antigen fused to a chaperone protein in an effective amount to increase lymph node uptake; improve an immune response or promote tolerance; or a combination relative to administering the peptide antigen alone and a pharmaceutically acceptable carrier.

20. The pharmaceutical composition of claim 19 further comprising an adjuvant.


SEQUENCE LISTING (30 sequences)

SEQ ID NO: 1 - protein, 20 aa, Artificial Sequence
  synthetic polypeptide
IDGPAGQAEPDRAHYNIVTF

SEQ ID NO: 2 - protein, 18 aa, Artificial Sequence
  synthetic polypeptide
RAYVSGIQNSVSANRSDP

SEQ ID NO: 3 - protein, 9 aa, Artificial Sequence
  synthetic polypeptide
TAPDNLGYM

SEQ ID NO: 4 - protein, 9 aa, Artificial Sequence
  synthetic polypeptide
TAPDNLGYA

SEQ ID NO: 5 - protein, 19 aa, Artificial Sequence
  synthetic polypeptide
DGPAGQAEPDRAHYNIVTF

SEQ ID NO: 6 - protein, 584 aa, Mus musculus
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 7 - protein, 238 aa, Artificial Sequence
  synthetic polypeptide
EPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 8 - protein, 238 aa, Artificial Sequence
  synthetic polypeptide
EPRVPITQNPCPPLKECPPCAAPDLLRGPSVFIFPPKIKDVLMISLSPMVTCVVVDVSEDDPDVQISWFVNNVEVHTAQTQTHREDYNSTLRVVSALPIQHQDWMSGKEFKCKVNNRARPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 9 - protein, 61 aa, Artificial Sequence
  synthetic polypeptide
ATVKYTYRGEEKRVDISKIKWVNRWGQHLAFKYDKGGGAAGYGWVSEKDAPKELLQMLEKR

SEQ ID NO: 10 - protein, 60 aa, Artificial Sequence
  synthetic polypeptide
ATVKFTYRGEEKQVDISKIKWVVRLGQVIMFKYDEGGGASGYGRVSEKDAPKELLQMLEK

SEQ ID NO: 11 - protein, 61 aa, Artificial Sequence
  synthetic polypeptide
ATVKFTYQGEEKQVDISKIKKVWRVGQMISFTYDEGGGATGRGAVSEKDAPKELLQMLEKQ

SEQ ID NO: 12 - protein, 4 aa, Artificial Sequence
  synthetic polypeptide
GSGS

SEQ ID NO: 13 - protein, 4 aa, Artificial Sequence
  synthetic polypeptide
GGGS

SEQ ID NO: 14 - protein, 12 aa, Artificial Sequence
  synthetic polypeptide
GGGSGGGSGGGS

SEQ ID NO: 15 - protein, 16 aa, Artificial Sequence
  synthetic polypeptide
GGGSGGGSGGGSGGGS

SEQ ID NO: 16 - protein, 8 aa, Artificial Sequence
  synthetic polypeptide
DYKDDDDK

SEQ ID NO: 17 - protein, 6 aa, Artificial Sequence
  synthetic polypeptide
YPYDVP

SEQ ID NO: 18 - protein, 10 aa, Artificial Sequence
  synthetic polypeptide
ILKKATAYIL

SEQ ID NO: 19 - protein, 10 aa, Artificial Sequence
  synthetic polypeptide
EQKLISEEDL

SEQ ID NO: 20 - protein, 20 aa, Artificial Sequence
  synthetic polypeptide
AVGALEGSRNQDWLGVPRQL

SEQ ID NO: 21 - protein, 20 aa, Artificial Sequence
  synthetic polypeptide
AVGALEGPRNQDWLGVPRQL

SEQ ID NO: 22 - protein, 147 aa, Mus musculus
MASLRLFLLCLAGLVFVSEAGPAGAGESKCPLMVKVLDAVRGSPAVDVAVKVFKKTSEGSWEPFASGKTAESGELHGLTTDEKFVEGVYRVELDTKSYWKTLGISPFHEFADVVFTANDSGHRHYTIAALLSPYSYSTTAVVSNPQN

SEQ ID NO: 23 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 24 - protein, 609 aa, Homo sapiens [sequence omitted]

SEQ ID NO: 25 - protein, 21 aa, Artificial Sequence
  synthetic polypeptide
MRVPAQLLGLLLLWLPGARCA

SEQ ID NO: 26 - protein, 127 aa, Artificial Sequence
  synthetic polypeptide
GPAGAGESKCPLMVKVLDAVRGSPAVDVAVKVFKKTSEGSWEPFASGKTAESGELHGLTTDEKFVEGVYRVELDTKSYWKTLGISPFHEFADVVFTANDSGHRHYTIAALLSPYSYSTTAVVSNPQN

SEQ ID NO: 27 - protein, 14 aa, Artificial Sequence
  synthetic polypeptide
GGGSHHHHHHGGGS

SEQ ID NO: 28 - protein, 585 aa, Artificial Sequence [sequence omitted]

SEQ ID NO: 29 - protein, 127 aa, Artificial Sequence
  synthetic polypeptide
GPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 30 - protein, 6 aa, Artificial Sequence
  synthetic polypeptide
HHHHHH